(19) United States
(12) Patent Application Publication
(10) Pub. No.: US 2005/0256757 A1
(43) Pub. Date: Nov. 17, 2005

(54) Method of manufacturing and method of marketing gender-specific absorbent articles having liquid-handling properties tailored to each gender

(76) Inventors: Alisa K. Sierra, Appleton, WI (US); Michael J. Niemeyer, Appleton, WI (US); Lawrence H. Sawyer, Neenah, WI (US)

Correspondence Address: KIMBERLY-CLARK WORLDWIDE, INC., 401 NORTH LAKE STREET, NEENAH, WI, 54956, US

(21) Appl. No.: 10/956,757

(22) Filed: Oct. 1, 2004

Related U.S. Application Data
(60) Provisional application No. 60/567,284, filed on Apr. 30, 2004.

(51) Int. Cl. G06F 17/60; A61F 13/15

(52) U.S. Cl. 705/10; 705/14

(57) ABSTRACT

Methods of manufacturing and methods of marketing gender-specific absorbent articles having liquid-handling properties tailored to each gender are disclosed. In certain embodiments, the properties of one or more fluid handling components used in disposable absorbent articles adapted for use by males is different than the properties of the corresponding component or components used in disposable absorbent articles adapted for use by females. Examples of the properties that can differ include permeability, capacity, void volume, basis weight, density, and the like. Examples of fluid handling components whose properties differ include surge materials, superabsorbent materials, absorbent cores, absorbent composites, and the like.

### RELATED APPLICATIONS

This application claims priority from U.S. Provisional Ser. No. 60/567,284, filed on Apr. 30, 2004.

### BACKGROUND

The present invention relates to methods of manufacturing and methods of marketing gender-specific disposable absorbent articles having liquid-handling properties tailored to each gender. In certain embodiments, the properties of one or more fluid handling components used in disposable absorbent articles adapted for use by males are different than the properties of the corresponding component or components used in disposable absorbent articles adapted for use by females. Examples of the properties that can differ include liquid permeability, absorbent capacity, void volume, basis weight, density, and the like. Examples of fluid handling components whose properties can differ include surge materials, superabsorbent materials, absorbent cores, absorbent composites, and the like.

Disposable absorbent articles are in widespread use in contemporary society. Examples of such articles include baby diapers, training pants, enuresis garments, and adult incontinence garments. Many disposable absorbent articles are marketed as “u nisex” products, and do not contain features specifically targeted to users of a particular gender. Other products are marketed as being specially designed for a particular gender, and include features adapted for users of a particular gender. For example, certain manufacturers offer both boy and girl disposable training pants, each of which includes printed graphics comprised of colors, scenes, and other indicia especially suitable for boys and girls, respectively.

In recognition of the fact that anatomical, physiological, and other differences exist between males and females that could impact the way bodily fluids (such as urine) are introduced into the absorbent article, efforts have been made to design products that recognize and address the impact of those differences. For example, the amount of cellulose wood pulp fiber, a material common to absorbent articles, used in a male product has been different than the amount of wood pulp fiber used in female products. In another example, the physical placement of a fluid handling component, such as a high-absorbency zone or a fluid intake layer, has been known to be varied as between male and female products. In still another example, the overall amount of high-absorbency polymeric material, commonly referred to as “s uperabsorbent” material or “superabsorbent” polymer, has been known to be varied as between male and female products.

Certain physical aspects of the materials commonly used in disposable absorbent articles are known to have relevance to the in-use performance of the articles. For example, the absorbent capacity of various components (i.e., the weight of fluid a component is able to absorb per unit weight) within the absorbent article affects the article's fluid-handling capabilities. Also, the permeability of various components (i.e., the ability of a component to allow liquid to pass through under specified conditions) within the article impacts performance as well. Other properties are believed to have relevance to in-use performance, such as the void volume present within a structure, the basis weight of a material, or the density of a material.

Certain materials, such as superabsorbent materials and fluid intake or “surge” materials, are useful in achieving high absorbency, low leakage, and relatively thin and discreet products. Despite the importance of these and other absorbency-related materials to modern absorbent articles, and despite the fact that anatomical and physiological differences exist between males and females, no effort has been made to optimize the properties of absorbent articles or of the components therein with respect to the different genders. While it has been known in the art to vary the overall amount of certain materials (such as superabsorbent polymer) or physical placement of materials (such as surge materials) between absorbent articles adapted specifically for males and females, there exists a need to optimize the selection of the structural and/or functional properties of the absorbency-related components used in products adapted for use by different genders so as to better meet the needs of the different genders.

### SUMMARY OF THE INVENTION

In response to the unmet needs in the art set forth above, new methods of manufacturing and of marketing disposable absorbent articles have been developed.

One aspect of the present invention relates to a method of marketing disposable absorbent articles for males and females. In one embodiment, the method of marketing comprises providing a plurality of packages of male absorbent articles adapted for use by males, each male absorbent article comprising a first superabsorbent polymer; providing a plurality of packages of female absorbent articles adapted for use by females, each female absorbent article comprising a second superabsorbent polymer, wherein the first superabsorbent polymer and the second superabsorbent polymer exhibit different average permeabilites as measured by the Free Swell Gel Bed Permeability method; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first superabsorbent polymer; providing a plurality of packages of female absorbent articles adapted for use by females, each female absorbent article comprising a second superabsorbent polymer, wherein the first superabsorbent polymer and the second superabsorbent polymer exhibit different average capacities as measured by the Centrifugal Retention Capacity method; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first superabsorbent polymer, the first superabsorbent polymer exhibiting a first average permeability and a first average capacity; providing a plurality of packages of female absorbent articles adapted for use by females, each female absorbent article comprising a second superabsorbent polymer, the second superabsorbent polymer exhibiting a second average permeability and a second average capacity, wherein the average permeabilities are measured by the Free Swell Gel Bed Permeability method, and the average capacities are measured by the Centrifugal Retention Capacity method, wherein a first ratio of the first average permeability to the first average capacity is higher than a second ratio of the second average permeability to the second average capacity; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first superabsorbent polymer; providing a plurality of packages of female absorbent articles adapted for use by females, each female absorbent article comprising a second superabsorbent polymer, wherein the first superabsorbent polymer exhibits a first average permeability as measured by the Free Swell Gel Bed Permeability method, and the second superabsorbent polymer exhibits an average permeability as measured by the Free Swell Gel Bed Permeability method, wherein a ratio of the first average permeability to the second average permeability is at least about 1.2; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first absorbent core; providing a plurality of packages of female absorbent articles adapted for use by females, each female absorbent article comprising a second absorbent core, wherein the first absorbent core and the second absorbent core exhibit different average permeabilities as measured by the Free Swell Absorbent Structure Permeability Test; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first surge material; providing a plurality of packages of female absorbent articles adapted for use by females, each female absorbent article comprising a second surge material, wherein the first surge material and the second surge material have different basis weights; and positioning the plurality of packages of male absorbent articles near the plurality of packages of female absorbent articles in a retail outlet.

In another embodiment, the method of marketing comprises providing a plurality of packages of male absorbent articles adapted for use by males, each male absorbent article comprising a first absorbent composite; providing a plurality of packages of female absorbent articles adapted for use by females, each female absorbent article comprising a second absorbent composite, wherein the first absorbent composite and the second absorbent composite exhibit different average void volumes as measured by the Void Volume Test; and positioning the plurality of packages of male absorbent articles near the plurality of packages of female absorbent articles in a retail outlet.

Another aspect of the present invention relates to a method of manufacturing disposable absorbent articles for males and females. In one embodiment, the method of manufacturing comprises assembling a plurality of male absorbent articles adapted for use by males, each male absorbent article comprising a first superabsorbent polymer; assembling a plurality of female absorbent articles adapted for use by females, each female absorbent article comprising a second superabsorbent polymer, wherein the first superabsorbent polymer and the second superabsorbent polymer exhibit different average permeabilities as measured by the Free Swell Gel Bed Permeability method; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

In another embodiment, the method of manufacturing comprises assembling a plurality of male absorbent articles adapted for use by males, each male absorbent article comprising a first superabsorbent polymer; assembling a plurality of female absorbent articles adapted for use by females, each female absorbent article comprising a second superabsorbent polymer, wherein the first superabsorbent polymer and the second superabsorbent polymer exhibit different average capacities as measured by the Centrifugal Retention Capacity method; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

In another embodiment, the method of manufacturing comprises assembling a plurality of male absorbent articles adapted for use by males, each male absorbent article comprising a first superabsorbent polymer; assembling a plurality of female absorbent articles adapted for use by females, each female absorbent article comprising a second superabsorbent polymer, wherein the first superabsorbent polymer exhibits a first average permeability as measured by the Free Swell Gel Bed Permeability method, and the second superabsorbent polymer exhibits an average permeability as measured by the Free Swell Gel Bed Permeability method, wherein a ratio of the first average permeability to the second average permeability is at least about 1.2; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

In another embodiment, the method of manufacturing comprises assembling a plurality of packages of male absorbent articles adapted for use by males, each male absorbent article comprising a first absorbent core; assembling a plurality of packages of female absorbent articles adapted for use by females, each female absorbent article comprising a second absorbent core, wherein the first absorbent core exhibits a first average permeability as measured by the Free Swell Absorbent Structure Permeability Test, and the second absorbent core exhibits a second average permeability as measured by the Free Swell Absorbent Structure Permeability Test, wherein a ratio of the first average permeability to the second average permeability is at least about 1.1; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

In another embodiment, the method of manufacturing comprises assembling a plurality of packages of male absorbent articles adapted for use by males, each male absorbent article comprising a first surge material having a first basis weight; assembling a plurality of packages of female absorbent articles adapted for use by females, each female absorbent article comprising a second surge material having a second basis weight, wherein a ratio of the first basis weight to the second basis weight is at least about 1.1; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

In another embodiment, the method of manufacturing comprises assembling a plurality of packages of male absorbent articles adapted for use by males, each male absorbent article comprising a first absorbent composite having a first void volume; assembling a plurality of packages of female absorbent articles adapted for use by females, each female absorbent article comprising a second absorbent composite having a second void volume, wherein a ratio of the first void volume to the second void volume is at least about 1.1; placing the plurality of male absorbent articles into a first package, wherein the first package includes indicia denoting that the absorbent articles therein are adapted for use by males; and placing the plurality of female absorbent articles into a second package, wherein the second package includes indicia denoting that the absorbent articles therein are adapted for use by females.

### DRAWINGS

The foregoing and other features and aspects of the present invention and the manner of attaining them will become more apparent, and the invention itself will be better understood by reference to the following description, appended claims and accompanying drawings, where:

FIG. 1 qualitatively illustrates the relationship between capacity and permeability of typical superabsorbent polymers.

FIG. 2 illustrates a cross-section of a representative apparatus for conducting a Gel Bed Permeability Test;

FIG. 3 is a section taken in the plane of line 3-3 of FIG. 2;

FIG. 4 illustrates a cross-section of an apparatus for conducting a Liquid Saturation Capacity Test;

FIGS. 5-8 illustrate the results achieved when a set of experimental products suitable for use in conjunction with the present invention were quantitatively tested; and

FIGS. 9-12 illustrate the results achieved when an alternative set of experimental products suitable for use in conjunction with the present invention were quantitatively tested; and

FIG. 13 representatively illustrates a training pant suitable for use in particular embodiments of the present invention.

### DETAILED DESCRIPTION OF PARTICULAR EMBODIMENTS

Disposable absorbent articles, such as diapers, training pants, and incontinence products, containing high-performance absorbent materials have been successful, and in recent years, market demand has increased for thinner, more absorbent and more comfortable disposable absorbent articles. Certain measurable physical properties of various components or structures contained within disposable absorbent articles have been found to have some relation to the performance of the absorbent articles.

One such property is the absorbent capacity of certain components or structures. The capacity of a component or structure refers generally to the amount of fluid (typically a liquid) that a particular mass or volume of that component or structure can absorb and retain under a particular set of conditions. Such components or structures include, for example, superabsorbent material; wood pulp fluff; a superabsorbent/wood pulp absorbent core; fluid intake layers; or the collective absorbent composite of each of the absorbency/fluid-handling materials with the absorbent article. “A bsorbent core” is intended to include superabsorbent polymer, wood pulp, any binder materials which provide structural integrity to the superabsorbent polymer/wood pulp matrix, and other materials whose primary function is to permanently retain/store fluid, but is not intended to include the topsheet, surge/intake layers, or core wrap materials. “Absorbent composite” is intended to include all materials and layers which assist in the transport, distribution, and retention of fluid, including but not limited to the topsheet, fluid-intake/surge layers, wood pulp, superabsorbent polymer, and core wrap materials, but is not intended to include generally liquid-impermeable materials, such as backsheets and containment flaps. Each of these materials is discussed more fully below. With respect to superabsorbent material in particular, due to the fact that market demand has in recent years increased for thinner, more absorbent disposable absorbent articles, it is generally desirable that the superabsorbent polymer used in such products exhibit a large absorbent capacity-to-mass ratio. In this way, relatively smaller amounts of superabsorbent polymer may be used while maintaining relatively higher absorbent capacities.

Another measurable physical property believed to be indicative of the fitness of certain components and structures for use in disposable absorbent articles is permeability. The permeability of an element refers generally to the ability of a fluid (typically a liquid) under a particular set of conditions to pass through the element. For example, one can quantify the permeability of surge materials, superabsorbent polymers, absorbent cores, or entire absorbent composites. For instance, the permeability of an absorbent core can be quantified by measuring the rate at which fluid passes through a swollen specimen of the absorbent core. Similarly, the permeability of a superabsorbent material can be quantified by measuring the amount of fluid that a swollen specimen of superabsorbent material can pass.

Frequently, with the aim of obtaining thin, highly absorbent disposable absorbent articles, the proportion of superabsorbent material in relation to other materials of the absorbent core is increased. There are, however, practical limits to increasing the proportion of superabsorbent particles in the absorbent core. If the concentration of superabsorbent material is too high, for example, gel blocking and/or capillary blocking can occur. When particles of superabsorbent material distributed through an absorbent core are exposed to liquid, they swell as they absorb the liquid, forming a gel. As adjacent particles of superabsorbent material swell, they can form a barrier to free liquid not immediately absorbed by the particles of superabsorbent material. As a result, access by the liquid to unexposed particles of superabsorbent material may be blocked by the swollen (gelled) particles of superabsorbent material. When gel blocking occurs, liquid pooling, as opposed to absorption, takes place in the absorbent core. As a result, large portions of the absorbent core may remain unused, and failure of the absorbent core can occur, typically in the form of leakage. Gel blocking caused by high concentrations of particles of superabsorbent material can result in reduced permeability of the absorbent core under pressures normally encountered during use of the disposable absorbent article. Thus, it is believed desirable for superabsorbent polymers used in disposable absorbent articles to exhibit a relatively high permeability, even at relatively high concentrations of superabsorbent polymer, thereby maintaining an adequate amount of capillary flow during use. Moreover, high levels of superabsorbent-particles, especially granular particles, can often lead to lower absorbent core integrity

Yet another physical property believed to have relevance to the fluid-handling properties of absorbent articles is void volume. “V oid volume” means free space within the absorbent structure available to accommodate incoming waste liquid. Stated more generally, it refers to the open space within a material that is not occupied by a solid or liquid component. It is believed that the amount of void volume within an absorbent structure, such as an absorbent core or an absorbent composite, is linked to the ability of the structure to receive, distribute, and store fluid. Void volume can be affected by a variety of factors, such as the size and stiffness of the constituent elements of the structure; the basis weight and density of the elements which provide void volume; the compressive forces to which the structure is subjected during processing, packaging, and use; and the like.

In summary, it is believed that the capacity, the permeability, the void volume, the basis weight, and the density of various materials and structures contained within disposable absorbent articles are closely connected with the in-use performance of such articles. Moreover, the inventors' research indicates that each of these factors has implications for the performance of male products different from the implications for the performance of female products. Notably, it has been discovered that these characteristics of the various absorbency-related materials and structures within the absorbent article have an impact on the performance of products used by males that is different than their impact on the performance of products used by females. For example, it has been discovered that the permeability characteristics of the absorbent core and the void volume characteristics of the absorbent composite each have a higher relevance to the performance of products used by males than to the performance of products used by females. Proper design and choice of the materials and structures for use in male and female absorbent articles in accordance with the present invention makes it possible to optimize the fluid-handling properties that are key performance drivers for the respective genders in a more efficient and cost-effective manner. Although the principles of the present invention are believed to apply with equal force to a variety of the absorbency-related materials or absorbency-related structures present within absorbent articles, the following illustrative embodiments and examples shall focus on the manipulation of superabsorbent material characteristics and surge material characteristics within absorbent articles, as well as the overall absorbent core and absorbent composite characteristics.

As used herein, the phrase “abs orbent article” refers to devices that absorb and contain body fluids, and more specifically, refers to devices that are placed against or near the skin to absorb and contain urine discharged from the body. The term “dis posable” is used herein to describe absorbent articles that are not intended to be laundered or otherwise restored or reused as an absorbent article after a single use. Examples of such disposable absorbent articles include, but are not limited to diapers, training pants, enuresis garments, feminine hygiene pads, and adult incontinence products.

Disposable absorbent articles such as those listed above typically include a fluid pervious topsheet, a liquid impervious backsheet joined to the topsheet and an absorbent body positioned between the topsheet and the backsheet. Disposable absorbent articles and components thereof, including the topsheet, backsheet, absorbent body and any individual layers of these components, generally have a body-facing surface and a garment-facing surface. As used herein, “body-facing surface” refers to that surface of the article or component which is intended to be worn toward or placed adjacent to the body of the wearer, while the “garment-facing surface” is on the opposite side and is intended to be worn toward or placed adjacent to the wearer's garments when the disposable absorbent article is worn.

The liquid impermeable backsheet can be both liquid and vapor impermeable, or can be liquid impermeable and vapor permeable. The liquid impermeable backsheet is desirably manufactured at least in part from a thin plastic film, although other flexible liquid impermeable materials can also be used. The liquid impermeable backsheet prevents waste material from wetting articles, such as bedsheets and clothing, as well as the wearer and caregiver. The liquid impermeable backsheet can include additional materials, such as cloth-like nonwoven materials well known in the art.

The topsheet is desirably compliant, soft feeling, and non-irritating to the user's skin. Further, the topsheet can be less hydrophilic than the absorbent body, to present a relatively dry surface to the wearer and permit liquid to readily penetrate through its thickness. Alternatively, the topsheet can be more hydrophilic or can have essentially the same affinity for moisture as the absorbent body to present a relatively wet surface to the wearer to increase the sensation of being wet. This wet sensation can be useful to signal to the user that the product has been insulted, such as during toilet training or for a person suffering from incontinence.

As discussed above, the absorbent body may be located between the liquid impermeable backsheet and the topsheet. The absorbent body can be any structure which is generally compressible, conformable, non-irritating to the child's skin, and capable of absorbing and retaining liquids and certain body wastes, and may be manufactured in a wide variety of sizes and shapes, and from a wide variety of liquid absorbent materials commonly used in the art. The absorbent body further includes superabsorbent polymer, as described in more detail below.

The absorbent article can also incorporate other materials or components designed primarily to receive, temporarily store, and/or transport liquid to different regions of the absorbent body, thereby maximizing the absorbent capacity of the absorbent body. One suitable additional component is commonly referred to as a liquid intake layer (alternatively referred to as an acquisition or surge layer), which can comprise a material having a basis weight of about 10 to about 300 grams per square meter, and in particular embodiments comprises a through-air-bonded-carded web of a homogenous blend of 60 percent 3 denier type T-256 bicomponent fiber comprising a polyester core/polyethylene sheath and 40 percent 6 denier type T-295 polyester fiber, both commercially available from Kosa Corporation of Salisbury, N.C., U.S.A. Additional examples of surge materials suitable for use in conjunction with the present invention include those disclosed in U.S. Pat. No. 5,486,166 issued to Bishop et al. and assigned to Kimberly-Clark Corporation and U.S. Pat. Nos. 5,820,973 and 5,879,343 issued to Dodge II et al. and assigned to Kimberly-Clark Worldwide, Inc., each of which is hereby incorporated by reference to the extent consistent herewith.

Absorbent articles employed in conjunction with the present invention can have front and back side panels disposed on each side of the absorbent body. The side panels can be permanently attached along seams to one or more components in the central section of the article, such as the topsheet and/or backsheet, using attachment means known to those skilled in the art such as adhesive, thermal or ultrasonic bonding. Alternatively, the side panels can be formed as an integral portion of one or more components of the article, such as a generally wider portion of the backsheet and/or topsheet. The front and back side panels can be permanently attached together, or be releasably connected with one another such as by a fastening system. The side panels suitably, although not necessarily, comprise an elastic material.

Any of the materials or components mentioned above can be non-stretchable, stretchable but non-elastomeric, or both stretchable and elastomeric. Examples of disposable absorbent articles suitable for use in conjunction with the present invention, and examples of the materials and components referenced above, include those disclosed in U.S. Pat. No. 6,645,190 issued to C. P. Olson et al., the entirety of which is hereby incorporated by reference to the extent consistent herewith.

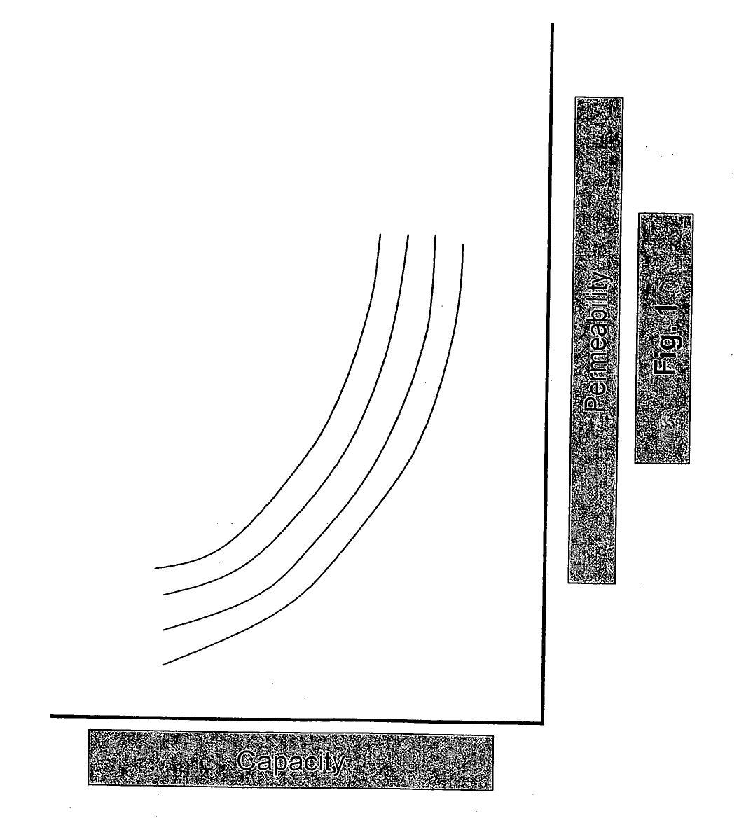

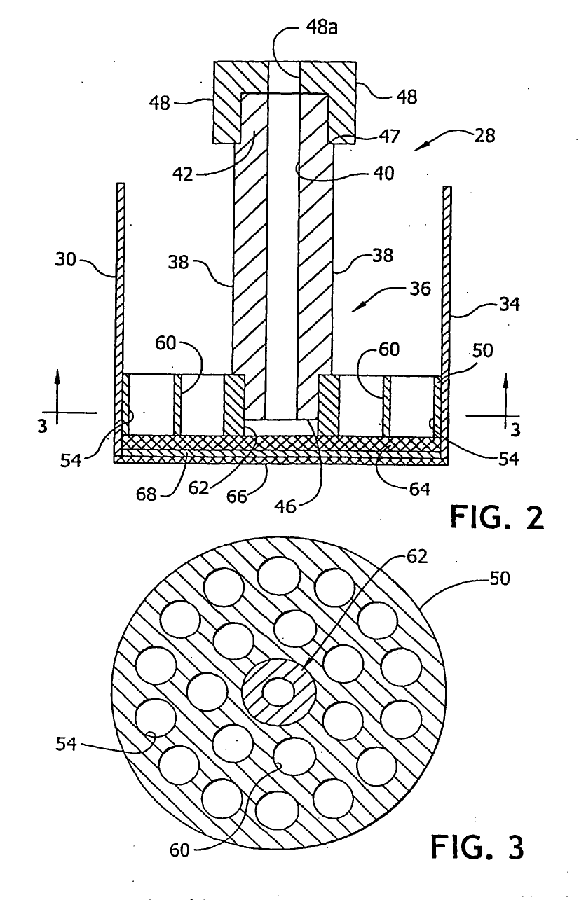

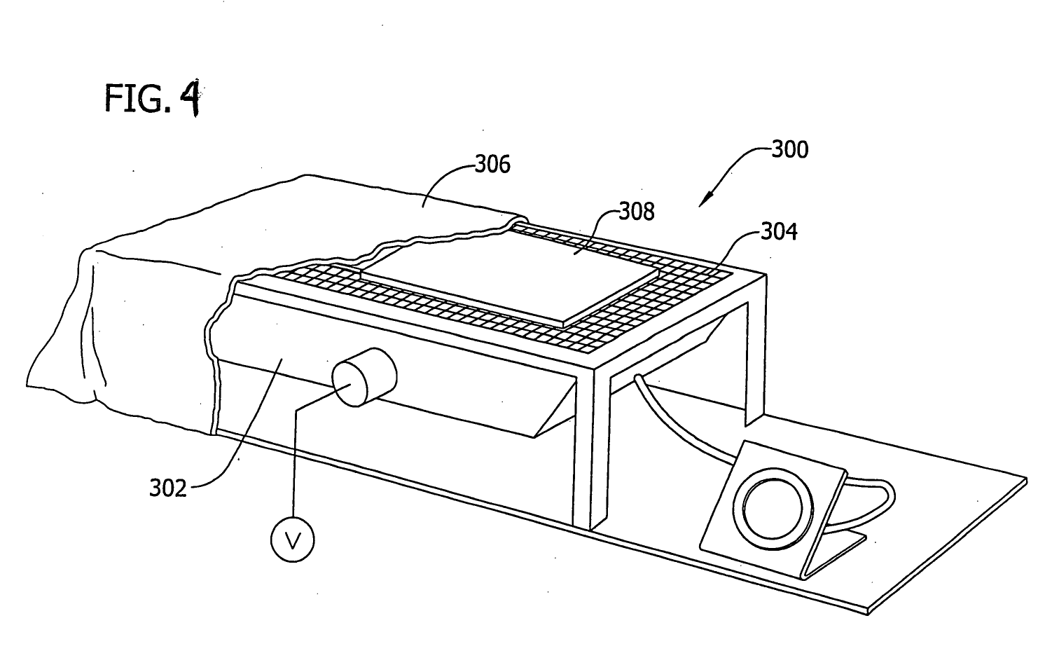

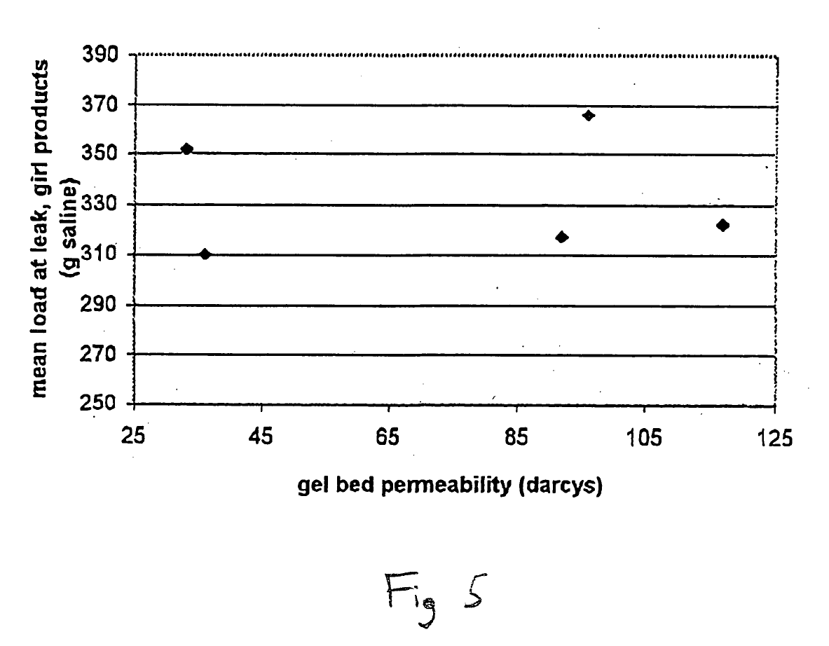

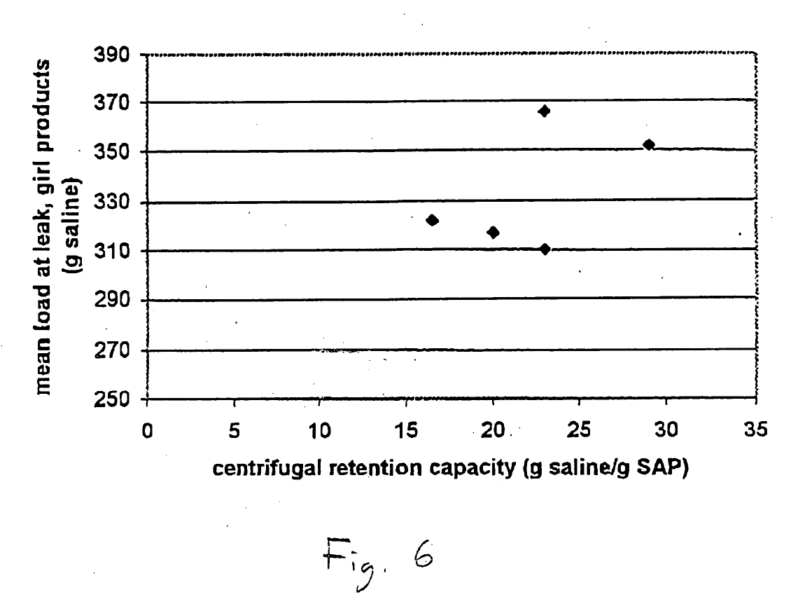

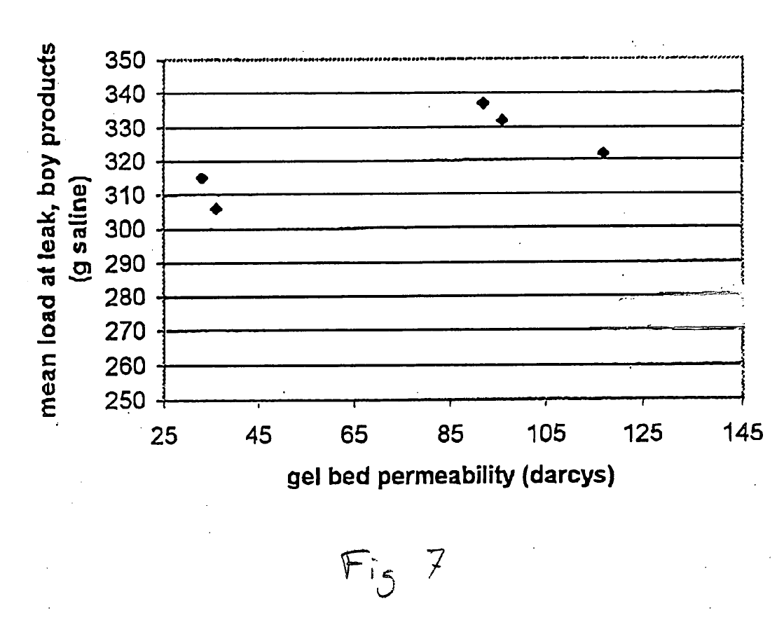

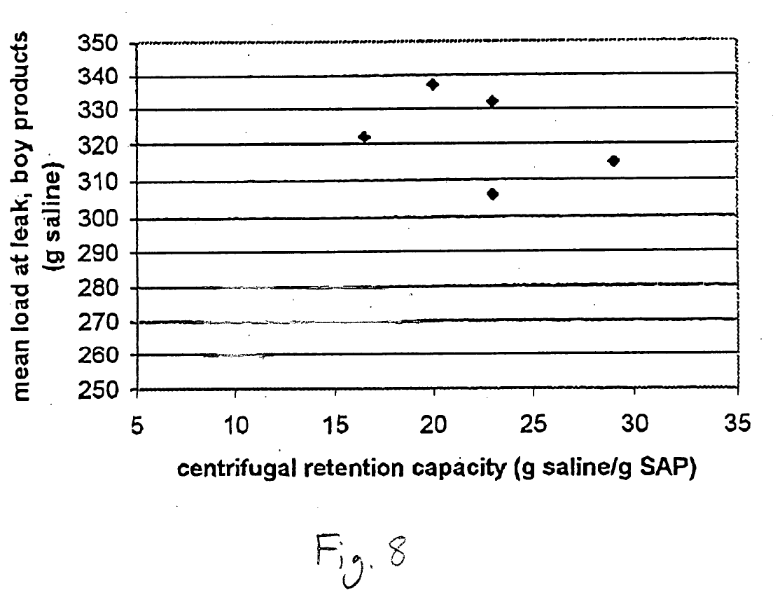

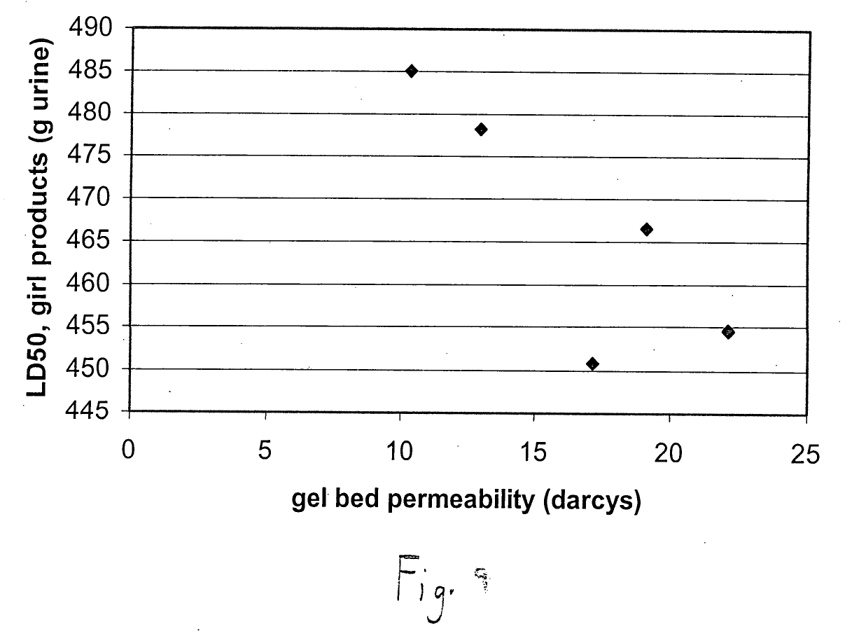

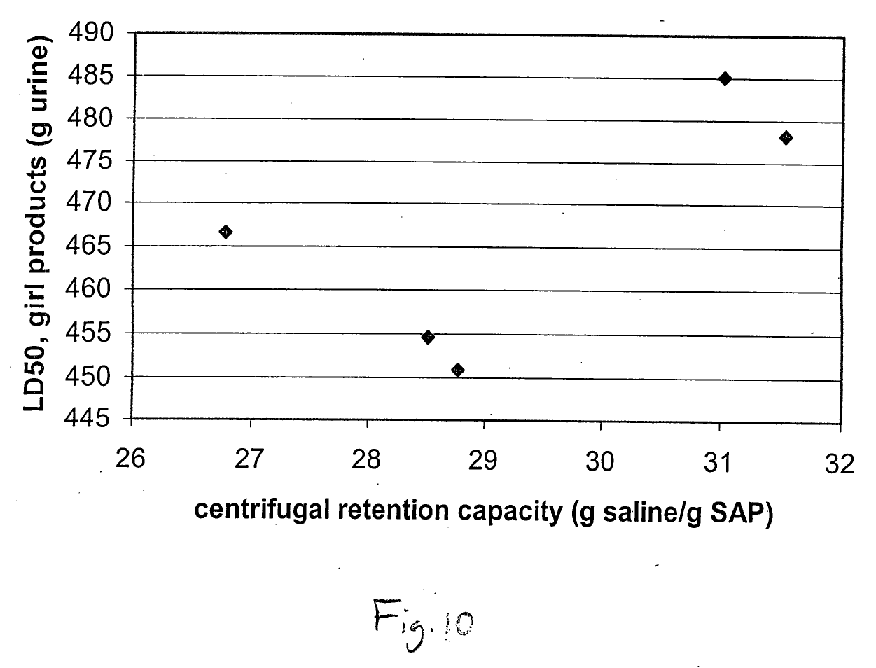

As alluded to above, it is believed that both the capacity and the permeability of a superabsorbent polymer are directly proportional to the efficacy of the superabsorbent polymer for use in disposable absorbent products. Unfortunately, with respect to superabsorbent material, one may generally only be increased at the expense of the other. In other words, superabsorbent polymers designed to deliver a relatively high absorbent capacity may lack optimal permeability, and superabsorbent polymers designed to deliver relatively high permeability may lack optimal capacity characteristics. Moreover, efforts to simultaneously increase both the capacity and permeability of a superabsorbent polymer generally increase its cost. Therefore, it can to some degree be necessary to balance, or “compromise,” the two factors when selecting the proper superabsorbent polymer for use in disposable absorbent articles. FIG. 1 representatively and qualitatively illustrates this capacity-to-permeability relationship which different families of conventional superabsorbent materials would typically exhibit. A particular “fa mily” of superabsorbent material is intended to refer to a group of materials whose capacity-to-permeability relationships vary, but whose basic chemistry and/or process of manufacture is the same or nearly the same. Different “fam ilies” would have different basic chemistries and/or processes of manufacture. Each curve in FIG. 1 is intended to representatively illustrate a range of capacity-to-permeability relationships within a particular family.

It has been discovered that certain superabsorbent polymers are more suitable for use in absorbent articles used by males, and that certain superabsorbent polymers are more suitable for use in absorbent articles used by females. In particular, the inventors, through research and development efforts, have discovered that superabsorbent polymer permeability has a much higher relevance to the performance of products used by males than to the performance of products used by females. Consequently, the target balance, or “compromise” as between optimization of capacity and optimization of permeability is different for male product applications than for female product applications. For example, the inventors' research has revealed that for a given product type and size, a superabsorbent polymer used in products worn by females can exhibit properties further to the upper left on any of the graphs shown in FIG. 1 than superabsorbent polymers used in products worn by males, and that such an arrangement offers an optimal balance as among female performance, male performance, and polymer cost. Alternatively, referring again to FIG. 1, it would be advantageous to, at a given capacity, select a superabsorbent polymer for use in a male product from a family of materials qualitatively represented by a curve relatively further to the right of the graph than a curve from which a superabsorbent polymer for use in a female product would be selected.

In one aspect, the present invention relates to a method of manufacturing disposable absorbent articles for males and females. One embodiment of the method includes assembling a plurality of male absorbent articles adapted for use by males, each male absorbent article comprising a first superabsorbent polymer, and further includes assembling a plurality of female absorbent articles adapted for use by females, each female absorbent article comprising a second superabsorbent polymer. Methods of absorbent article assembly per se are well known in the art, and a detailed recitation of the specific technical aspects of such methods is not necessary for an understanding of the present invention. Processes suitable for the assembly of absorbent articles that include superabsorbent polymers are disclosed in U.S. Pat. Nos. 6,652,167 and 6,514,187 issued to Coenen et al., each of which is hereby incorporated by reference to the extent consistent herewith.

In certain embodiments, superabsorbent polymer employed in conjunction with the present invention is in particulate form. By “particle,” “p articles,” “pa rticulate,” “particulates” and the like, it is meant that a material is generally in the form of discrete units. The particles can include granules, pulverulents, powders or spheres. Thus, the particles can have any desired shape such as, for example, cubic, rod-like, polyhedral, spherical or semi-spherical, rounded or semi-rounded, angular, or irregular. Shapes having a large greatest dimension/smallest dimension ratio, like needles, flakes and fibers, are also contemplated for use herein. The use of “pa rticle” or “par ticulate” may also describe an agglomeration including more than one particle, particulate or the like.

As used herein, “s uperabsorbent polymer,” “superabsorbent material,” “superabsorbent materials” and the like are intended to refer to a water-swellable, water-insoluble organic or inorganic material capable, under the most favorable conditions, of absorbing at least about 10 times its weight and, desirably, at least about 15 times its weight in an aqueous solution containing 0.9 weight percent of sodium chloride. Such materials include, but are not limited to, hydrogel-forming polymers that are alkali metal salts of: poly(acrylic acid); poly(methacrylic acid); copolymers of acrylic and methacrylic acid with acrylamide, vinyl alcohol, acrylic esters, vinyl pyrrolidone, vinyl sulfonic acids, vinyl acetate, vinyl morpholinone and vinyl ethers; hydrolyzed acrylonitrile grafted starch; acrylic acid grafted starch; maleic anhydride copolymers with ethylene, isobutylene, styrene, and vinyl ethers; polysaccharides such as carboxymethyl starch, carboxymethyl cellulose, methyl cellulose, and hydroxypropyl cellulose; poly(acrylamides); poly(vinyl pyrrolidone); poly(vinyl morpholinone); poly(vinyl pyridine); and copolymers and mixtures of any of the above and the like. The hydrogel-forming polymers are suitably lightly cross-linked to render them substantially water-insoluble. Cross-linking may be achieved by irradiation or by covalent, ionic, van der Waals, or hydrogen bonding interactions, for example. A suitable superabsorbent material is a lightly cross-linked hydrocolloid. Specifically, a more suitable superabsorbent material is a partially neutralized polyacrylate salt. Superabsorbent materials useful in the present invention are generally available from various commercial vendors, such as, for example, BASF Corporation of Portsmouth, Virginia, U.S.A., or Stockhausen Inc. of Greensboro, N.C., U.S.A.

Suitably, the superabsorbent material is in the form of particles which, in the unswollen state, have maximum diameters ranging between about 50 and about 1,000 microns; suitably, between about 100 and about 800 microns; more suitably, between about 200 and about 650 microns; and most suitably, between about 300 and about 600 microns, as determined by sieve analysis according to American Society for Testing Materials Test Method D-1921. It is understood that the particles of superabsorbent material may include solid particles, porous particles, or may be agglomerated particles including many smaller particles agglomerated into particles falling within the described size ranges.

It should be noted that as superabsorbent polymer is incorporated into absorbent articles during the absorbent-article manufacturing process, the superabsorbent polymer can be subject to various forces which can damage the material or otherwise change its physical properties. Therefore, superabsorbent polymer as it exists prior to conversion into absorbent articles (hereinafter referred to as “virg in SAP” or “virgin SAM”) can exhibit different performance characteristics than superabsorbent polymer as it exists subsequent to conversion in absorbent articles (hereinafter “reclaim SAP” or “r eclaim SAM”). Certain features and embodiments of the invention are herein described in terms of various quantifiable functional or performance parameters exhibited by either virgin SAM, reclaim SAM, or both. Also, although the following description of particular embodiments focuses on the method-of-manufacturing aspect of the present invention, the various configurations are equally suitable for use in conjunction with the method-of-marketing aspect of the present invention.

One analytical method suitable for quantifying the permeability of superabsorbent polymers is the Free Swell Gel Bed Permeability (GBP) test, set forth in detail below. Superabsorbent polymers employed in conjunction with the present invention typically have a Free Swell GBP, as tested in a virgin or reclaim condition, of no less than 5; alternatively, no less than 10; or alternatively, no less than 20 Darcies. In addition, superabsorbent polymers employed in the present invention typically have a Free Swell GBP, as tested in a virgin or reclaim condition, of no more than 1000; alternatively, no more than 500; alternatively, no more than 200; or alternatively, no more than 150 Darcies. Thus, a superabsorbent polymer employed with the present invention can in particular embodiments have a Free Swell GBP, as tested in a virgin or reclaim condition, ranging between no less than 5 Darcies up to no more than 1000 Darcies, although the Free Swell GBP of a superabsorbent polymer employed with the present invention can vary according to the general design and intended use of the superabsorbent.

In particular embodiments of the method of manufacturing of the present invention, a first superabsorbent polymer employed in male absorbent articles and a second superabsorbent polymer employed in female absorbent articles exhibit different average permeabilities. “Di fferent average permeabilities” means that the average permeabilities in question are statistically significantly different at a 90% level of confidence. In such embodiments, the average permeability of the first superabsorbent polymer can be higher than the average permeability of the second superabsorbent polymer. In particular embodiments, the average Free Swell GBP of the first superabsorbent polymer, as tested in a virgin or reclaim condition, is at least about 10 Darcies, more particularly at least about 40 Darcies, more particularly at least about 60 Darcies, more particularly at least about 80 Darcies, and more particularly at least about 100 Darcies. In particular embodiments, the average Free Swell GBP of the second superabsorbent polymer, as tested in a virgin or reclaim condition, is at most about 60 Darcies, more particularly at most about 40 Darcies, more particularly at most about 30 Darcies, still more particularly at most about 20 Darcies, and still more particularly at most about 5 Darcies.

One means by which to quantify the absorbent capacity of a superabsorbent polymer is to measure its ability to retain liquid while subjected to a centrifugal force. One analytical method recognized in the art as being suitable for quantifying the ability of a superabsorbent polymer to retain liquid while subject to a centrifugal force is the Centrifuge Retention Capacity (CRC) test, set forth in detail below. The various preferred values and ranges of CRC values set forth below apply to both virgin SAM and reclaimed SAM. Superabsorbent polymers employed as part of the present invention typically have a CRC of no less than 15; alternatively, no less than 20; alternatively, no less than 25; or, alternatively, no less than 30 g/g. In addition, a superabsorbent polymer employed in the present invention typically has a CRC of no more than 60; alternatively, no more than 50; alternatively, no more than 40; or, alternatively, no more than 30 g/g. Thus, a superabsorbent polymer employed with the present invention can in particular embodiments have a CRC ranging between no less than 15 g/g up to no more than 60 g/g, although the CRC of a superabsorbent polymer employed with the present invention can vary according to the general design and intended use of the superabsorbent.

In particular embodiments of the method of manufacturing of the present invention, a first superabsorbent polymer employed in male absorbent articles and a second superabsorbent polymer employed in female absorbent articles exhibit different average capacities. “D ifferent average capacities” means that the average capacities in question are statistically significantly different at a 90% level of confidence. In particular embodiments, the average capacity of the first superabsorbent polymer is lower than the average capacity of the second superabsorbent polymer. In particular embodiments, the average CRC of the first superabsorbent polymer is at most about 28 g/g, and more particularly at most about 20 g/g. In particular embodiments, the average CRC of the second superabsorbent polymer is at least about 27 g/g, and more particularly at most about 32 g/g.

In the method of manufacturing of the present invention, a first superabsorbent polymer employed in male absorbent articles exhibits a first average permeability and a first average capacity, and a second superabsorbent polymer employed in female absorbent articles exhibits a second average permeability and a second average capacity. In particular embodiments, the ratio of the first average permeability to the first average capacity is higher than the ratio of the second average permeability to the second average capacity. In particular embodiments, the ratio of the first average permeability to the first average capacity is at least about 1.6 Darcy-g/g, and more particularly at least about 2.1 Darcy-g/g. In particular embodiments, the ratio of the second average permeability to the second average capacity is at least about 1.0 Darcy-g/g, and more particularly at least about 1.4 Darcy-g/g. Furthermore, in particular embodiments, the ratio of the first average permeability to the first average capacity is at least about 10% greater, more particularly at least about 30% greater, more particularly at least about 50% greater, more particularly at least about 70% greater, more particularly at least about 90% greater, and more particularly at least about 110% greater than the ratio of the second average permeability to the second average capacity. In such embodiments, the average permeabilities can be measured by the Free Swell Gel Bed Permeability method, and the average capacities can be measured by the Centrifugal Retention Capacity method. Either virgin or reclaim SAM can be used for either analysis.

In particular embodiments of the method of manufacturing of the present invention, a first superabsorbent polymer employed in male absorbent articles exhibits a first average permeability, and a second superabsorbent polymer employed in female absorbent articles exhibits a second average permeability, and the ratio of the first average permeability to the second average permeability is at least about 1.2, more particularly at least about 1.6, and more particularly at least about 2.0. In such embodiments, the average permeabilities can be measured by the Gel Bed Permeability method, and either virgin SAM or reclaim SAM can be used.

Another embodiment of the method of manufacturing of the present invention includes assembling a plurality of male absorbent articles absorbent article adapted for use by males, each male absorbent article comprising a first absorbent core, and further includes assembling a plurality of female absorbent articles adapted for use by females, each female absorbent article comprising a second absorbent core. One analytical method suitable for quantifying the permeability of absorbent cores is the Absorbent Structure Permeability (ASP) test, set forth in detail below. The ASP test can be conducted in either Free Swell or 0.3 psi mode. Absorbent cores employed in conjunction with the present invention typically have a Free Swell ASP of no less than 5; alternatively, no less than 25; alternatively, no less than 50; or alternatively, no less than 100 Darcies. In addition, absorbent cores employed in the present invention typically have a Free Swell ASP of no more than 100; alternatively, no more than 50; alternatively, no more than 25; or alternatively, no more than 5 Darcies. Absorbent cores employed in conjunction with the present invention typically have a 0.3 psi ASP of no less than 0.5; alternatively, no less than 5; alternatively, no less than 10; or alternatively, no less than 25 Darcies. In addition, absorbent cores employed in the present invention typically have a 0.3 psi ASP of no more than 25; alternatively, no more than 10; alternatively, no more than 5; or alternatively, no more than 0.5 Darcies. The ASP of an absorbent core employed with the present invention can vary according to the general design and intended use of the absorbent core.

In particular embodiments of the method of manufacturing of the present invention, the first absorbent core and the second absorbent core exhibit different average permeabilities. In such embodiments, the average permeability of the first absorbent core can be higher than the average permeability of the second absorbent core. In particular embodiments, the average Free Swell ASP of the first absorbent core is at least about 5 Darcies, more particularly at least about 25 Darcies, more particularly at least about 50 Darcies, and more particularly at least about 100 Darcies. In particular embodiments, the average ASP of the second absorbent core is at most about 100 Darcies, more particularly at most about 50 Darcies, more particularly at most about 25 Darcies, and more particularly at most about 5 Darcies. Additionally, in particular embodiments, the average 0.3 psi ASP of the first absorbent core is at least about 0.5 Darcies, more particularly at least about 5 Darcies, more particularly at least about 10 Darcies, and more particularly at least about 25 Darcies. In particular embodiments, the average ASP of the second absorbent core is at most about 25 Darcies, more particularly at most about 10 Darcies, more particularly at most about 5 Darcies, and more particularly at most about 0.5 Darcies. Furthermore, in particular embodiments of the method of manufacturing of the present invention, a first absorbent core employed in male absorbent articles exhibits a first average ASP, and a second absorbent core employed in female absorbent articles exhibits a second average ASP, and the ratio of the first average ASP to the second average ASP is at least about 1.1, more particularly at least about 1.2, and more particularly at least about 1.4. Such exemplary and desirable permeability ratios hold true for both Free Swell and 0.3 psi ASP characterizations.

In particular embodiments of the present invention, a first surge material having a first basis weight is employed in male absorbent articles, and a second surge material having a second, different basis weight is employed in female absorbent articles. The first and second surge materials can have the same or different chemical compositions. In such embodiments, first basis weight can but need not be higher than the second basis weight. In particular embodiments, the basis weight of the first surge material is at least about 40 grams per square meter, more particularly at least about 85 grams per square meter, and can in particular embodiment be at least about 100 grams per square meter. Furthermore, in particular embodiments, the basis weight of the second surge material is at most about 85 grams per square meter, more particularly at most about 75 grams per square meter, and more particularly at most about 30 grams per square meter. Additionally, in certain embodiments, the ratio of the basis weight of the surge material employed in male absorbent articles to the basis weight of the surge material employed in female absorbent articles is at least about 1.1, more particularly at least about 1.3, at yet more particularly at least about 1.5. Moreover, in certain embodiments of the method of manufacturing of the present invention, each male absorbent article includes a surge material, and each female absorbent article does not include a similar surge material.

Without wishing the following theory to limit the scope of the invention, it is believed that by in particular embodiments providing male absorbent articles with relatively more interstitial void volume into which fluid insults can flow, an array of male and female absorbent articles is provided in the most effective and efficient manner. For example, a relatively higher basis weight of surge material in male absorbent articles (relative to the basis weight of surge in female absorbent articles) provides a relatively higher amount of total interstitial void volume into which male urine insults can flow. Similarly and more broadly, a relatively higher amount of interstitial void volume in the absorbent composite of male absorbent articles (relative to the interstitial void volume in the absorbent composite of female absorbent articles) provides a relatively higher amount of total interstitial void volume into which male urine insults can flow. Thus, in particular embodiments of the present invention, a first absorbent composite employed in male absorbent articles exhibits a first overall void volume, and a second absorbent composite employed in female absorbent articles exhibits a second, different overall void volume. “D ifferent overall void volume” means that the void volumes in question are statistically significantly different at a 90% level of confidence. In such embodiments, the first void volume can be greater than the second void volume. In particular embodiments, the first void volume is at least about 20 cubic centimeters, more particularly at least about 60 cubic centimeters, and yet more particularly at least about 90 cubic centimeters. In particular embodiments, the second void volume is at most about 50 cubic centimeters, more particularly at most about 30, and still more particularly at most about 15 cubic centimeters. Additionally, in certain embodiments, the ratio of the first void volume to the second void volume is at least about 1.1, more particularly at least about 1.3, at yet more particularly at least about 1.5.

The method of manufacturing of the present invention can further include placing the plurality of male absorbent articles into a first package, wherein the first package includes indicia denoting that the absorbent articles therein are adapted for use by males, and further includes placing the plurality of female absorbent articles into a second package, wherein the second package includes indicia denoting that the absorbent articles therein are adapted for use by females. In particular embodiments, the packages of male absorbent articles can include indicia which denotes that the male absorbent articles include an absorbent material (such as, for example, a superabsorbent material) specifically adapted for use in male absorbent articles, and the packages of female absorbent articles can include indicia which denotes that the female absorbent articles include an absorbent material (such as, for example, superabsorbent material) specifically adapted for use in female absorbent articles. In particular embodiments of the method of the present invention, each male absorbent article includes a surge material, and no female absorbent articles include a surge material, due to the fact that surge materials have been found to have a greater relevance to the performance of male products.

### Test Methods

### Free Swell Gel Bed Permeability Test

As used herein, the Free Swell Gel Bed Permeability (GBP) Test determines the permeability of a swollen bed of gel particles (e.g., such as the absorbent composites or the non-coated superabsorbent material), under what is commonly referred to as “f ree swell” conditions. The term “fre e swell” means that the gel particles are allowed to swell without a restraining load upon absorbing test solution as will be described. A suitable apparatus for conducting the Gel Bed Permeability Test is illustrated in FIGS. 2 and 3 and indicated generally at 28. The test apparatus (28) includes a sample container, generally indicated at 30, and a piston, generally indicated at 36. The piston (36) comprises a cylindrical LEXAN shaft (38) having a concentric cylindrical hole (40) bored down the longitudinal axis of the shaft. Both ends of the shaft (38) are machined to provide upper and lower ends respectively designated 42, 46. A weight (48) rests on one end (42) and has a cylindrical hole (48a) bored through at least a portion of its center.

A circular piston head (50) is positioned on the other end (46) and is provided with a concentric inner ring of seven holes (60), each having a diameter of about 0.95 cm, and a concentric outer ring of fourteen holes (54), also each having a diameter of about 0.95 cm. The holes (54, 60) are bored from the top to the bottom of the piston head (50). The piston head (50) also has a cylindrical hole (62) bored in the center thereof to receive an end (46) of the shaft (38). The bottom of the piston head (50) may also be covered with a biaxially stretched 100 mesh stainless steel screen (64).

The sample container (30) includes a cylinder (34) and a 400 mesh stainless steel cloth screen (66) that is biaxially stretched to tautness and attached to the lower end of the cylinder. A gel particle sample (68) is supported on the screen (66) within the cylinder (34) during testing.

The cylinder (34) may be bored from a transparent LEXAN rod or equivalent material, or it may be cut from a LEXAN tubing or equivalent material, and has an inner diameter of about 6 cm (e.g., a cross-sectional area of about 28.27 cm²), a wall thickness of about 0.5 cm and a height of approximately 10 cm. Drainage holes (not shown) are formed in the sidewall of the cylinder (34) at a height of approximately 7.8 cm above the screen (66) to allow liquid to drain from the cylinder to thereby maintain a fluid level in the sample container at approximately 7.8 cm above the screen (66). The piston head (50) is machined from a LEXAN rod or equivalent material and has a height of approximately 16 mm and a diameter sized such that it fits within the cylinder (34) with minimum wall clearance but still slides freely. The shaft (38) is machined from a LEXAN rod or equivalent material and has an outer diameter of about 2.22 cm and an inner diameter of about 0.64 cm.

The shaft upper end (42) is approximately 2.54 cm long and approximately 1.58 cm in diameter, forming an annular shoulder (47) to support the weight (48). The weight (48) has an inner diameter of about 1.59 cm so that it slips onto the upper end (42) of the shaft (38) and rests on the annular shoulder (47) formed thereon. The weight (48) can be made from stainless steel or from other suitable materials resistant to corrosion in the presence of the test solution, which is 0.9 weight percent sodium chloride solution in distilled water. The combined weight of the piston (36) and weight (48) is approximately 596 grams (g), which corresponds to a pressure applied to the sample (68) of about 0.3 pounds per square inch (psi), or about 20.7 dynes/cm² (2.07 kPa), over a sample area of about 28.27 cm².

When the test solution flows through the test apparatus during testing as described below, the sample container (30) generally rests on a 16 mesh rigid stainless steel support screen (not shown). Alternatively, the sample container (30) may rest on a support ring (not shown) diametrically sized substantially the same as the cylinder (34) so that the support ring does not restrict flow from the bottom of the container.

To conduct the Gel Bed Permeability Test under “f ree swell” conditions, the piston (36), with the weight (48) seated thereon, is placed in an empty sample container (30) and the height is measured using a suitable gauge accurate to 0.01 mm with the platen removed. It is important to measure the height of each sample container (30) empty and to keep track of which piston (36) and weight (48) is used when using multiple test apparatus. The same piston (36) and weight (48) should be used for measurement when the sample (68) is later swollen following saturation.

The sample to be tested is prepared from particles which are prescreened through a U.S. standard 30 mesh screen and retained on a U.S. standard 50 mesh screen. As a result, the test sample includes particles sized in the range of about 300 to about 600 microns. The particles can be prescreened by hand or automatically. Approximately 0.9 grams of the sample is placed in the sample container (30), and the container, without the piston (36) and weight (48) therein, is then submerged in the test solution for a time period of about 60 minutes to saturate the sample and allow the sample to swell free of any restraining load.

At the end of this period, the piston (36) and weight (48) assembly is placed on the saturated sample (68) in the sample container (30) and then the sample container (30), piston (36), weight (48), and sample (68) are removed from the solution. The thickness of the saturated sample (68) is determined by again measuring the height from the bottom of the weight (48) to the top of the cylinder (34), using the same thickness gauge used previously provided that the zero point is unchanged from the initial height measurement.

The height measurement obtained from measuring the empty sample container (30), piston (36), and weight (48) is subtracted from the height measurement obtained after saturating the sample (68). The resulting value is the thickness, or height “H” of the swollen sample.

The permeability measurement is initiated by delivering a flow of the test solution into the sample container (30) with the saturated sample (68), piston (36), and weight (48) inside. The flow rate of test solution into the container is adjusted to maintain a liquid height of 7.8 cm above the bottom of the sample container. The quantity of solution passing through the sample (68) versus time is measured gravimetrically. Data points corresponding to the cumulative weight of the liquid passing through the sample are collected every second for at least twenty seconds once the fluid level has been stabilized to and maintained at 7.8 cm in height. The flow rate Q through the swollen sample (68) is determined in units of grams/second (g/s) by a linear least-square fit of fluid passing through the sample (68) (in grams) versus time (in seconds).

Permeability in cm² is obtained by the following equation:
K=[Q*H*Mu]/[A*Rho*P]
where K=Permeability (cm²), Q=flow rate (g/sec), H=height of sample (cm), Mu=liquid viscosity (poise) (approximately one centipoises for the test solution used with this Test), A=cross-sectional area for liquid flow (cm²), Rho=liquid density (g/cm³) (approximately one g/cm³, for the test solution used with this Test) and P=hydrostatic pressure (dynes/cm²) (normally approximately 7,652 dynes/cm²). The hydrostatic pressure is calculated from
   P=Rho*g*h
where Rho=liquid density (g/cm³), g=gravitational acceleration, nominally 981 cm/sec², and h=fluid height, e.g., 7.8 cm for the Free Swell Gel Bed Permeability Test described herein.

A minimum of three samples are tested and the results are averaged to determine the Gel Bed Permeability of the sample. The samples are tested at 23±1 degrees Celcius at 50±2 percent relative humidity.

### Centrifuge Retention Capacity Test

The Centrifuge Retention Capacity (CRC) Test measures the ability of the gel particles (e.g., such as the absorbent composites or the non-coated superabsorbent material) to retain liquid therein after being saturated and subjected to centrifugation under controlled conditions. The centrifugation is meant to remove the fluid which resides interstitially between the SAM particles. The resultant retention capacity is stated as grams of liquid retained per gram weight of the sample (g/g). The sample to be tested is prepared from particles which are prescreened through a U.S. standard 30 mesh screen and retained on a U.S. standard 50 mesh screen. As a result, the sample comprises particles sized in the range of about 300 to about 600 microns. The particles can be prescreened by hand or automatically and are stored in a sealed airtight container until testing.

The retention capacity is measured by placing 0.2±0.005 grams of the prescreened sample into a water-permeable bag that will contain the sample while allowing a test solution (0.9 weight/volume percent sodium chloride in distilled water) to be freely absorbed by the sample. A heat-sealable tea bag material, such as that available from Dexter Corporation of Windsor Locks, Conn., USA, as model designation 1234T heat sealable filter paper works well for most applications. The bag is formed by folding a 5-inch by 3-inch sample of the bag material in half and heat-sealing two of the open edges to form a 2.5-inch by 3-inch rectangular pouch. The heat seals should be about 0.25 inches inside the edge of the material. After the sample is placed in the pouch, the remaining open edge of the pouch is also heat-sealed. Empty bags are also made to serve as controls. Three samples (e.g., filled and sealed bags) are prepared for the test. The filled bags must be tested within three minutes of preparation unless immediately placed in a sealed container, in which case the filled bags must be tested within thirty minutes of preparation. The bags are placed between two TEFLON® coated fiberglass screens having 3 inch openings (Taconic Plastics, Inc., Petersburg, N.Y., USA) and submerged in a pan containing 1000 grams of the test solution at 23 degrees Celsius, making sure that the screens are held down until the bags are completely wetted. After wetting, the samples remain in the solution for about 30±1 minutes, at which time they are removed from the solution and temporarily laid on a non-absorbent flat surface. For multiple tests, the pan should be emptied and refilled with fresh test solution after 24 bags have been saturated in the pan.

The wet bags are then placed into the basket of a suitable centrifuge capable of subjecting the samples to a g-force of about 350. One suitable centrifuge is a Heraeus LaboFuge 400 having a water collection basket, a digital rpm gauge, and a machined drainage basket adapted to hold and drain the bag samples. Where multiple samples are centrifuged, the samples must be placed in opposing positions within the centrifuge to balance the basket when spinning. The bags (including the wet, empty bags) are centrifuged at about 1,600 rpm (e.g., to achieve a target g-force of about 350), for 3 minutes. The bags are removed and weighed, with the empty bags (controls) being weighed first, followed by the bags containing the samples. The amount of solution retained by the sample, taking into account the solution retained by the bag itself, is the CRC of the sample, expressed as grams of fluid per gram of sample. More particularly, the CRC is determined as:
CRC=(SB-EB-DS)/DS
where: SB=sample/bag weight after centrifuge;
EB=empty bag weight after centrifuge; and
DS=dry sample weight

The three samples are tested and the results are averaged to determine the CRC. The samples are tested at reasonable ambient conditions, such as 23±1 °C. at 50±2 percent relative humidity.

### Example of Method to Reclaim Superabsorbent

After a core of superabsorbent material and wood-pulp fluff is prepared, such as in a partially or fully assembled absorbent article, superabsorbent material can be recovered or “reclaimed” from the core by the following method. This approach may be helpful where there is a concern about possible modification of or damage to the superabsorbent during the preparation of cores.

The absorbent core is isolated from other product components, and any wrap sheet is removed. The core can be broken or torn apart into smaller sections, such as less than about 20 square centimeters (cm²) in size. The sections can be enclosed within a chamber that can nondestructively loosen and separate the components of the core. For example, the chamber may be equipped to provide simultaneous air agitation and a continuous passage of air through the chamber. The agitation can be designed to effectively break up the sections into their individual components of superabsorbent and fluff. The chamber can additionally include a porous support member for the sections of absorbent composite; the support member can prevent intact pieces of core from passing through into the collection area, but can permit passage of loose superabsorbent particles and fluff fibers. The rate of air passage through the chamber can be adjusted to entrain the lightest components (generally fluff fibers) and remove them from the chamber. Any remaining chunks of core material that are not broken up in the agitation can be removed. The granular superabsorbent remaining at the end of the process can be isolated from the bottom of the chamber, and treated in any manner desired for subsequent testing, such as sieving to obtain a given particle size fraction, or other treatment. It is also contemplated that the superabsorbent material can be separated from the other product components in other ways, such as manually shaking the absorbent core of the product, thereby mechanically and gravimetrically displacing the superabsorbent particles from the rest of the absorbent structure.

### Absorbent Structure Permeability Test

The Absorbent Structure Permeability Test is used to determine the permeability of the absorbent structure (such as an absorbent core), and more particularly a “z-direction” permeability of the absorbent structure based on liquid flow through the thickness of the structure. This test is substantially similar to the Free Swell Gel Bed Permeability Test set forth above, with the following noted exceptions. Referring back to FIGS. 2 and 3, instead of the cylinder height being about 5 cm, the cylinder height should be about 10 cm. Also, instead of particulate superabsorbent material being placed in the sample container, a circular absorbent structure sample 68 (e.g., either formed or otherwise cut from a larger absorbent structure), with any tissue wrap removed and having a cross-sectional diameter of about 6 cm is placed in the sample container 30 at the bottom of the cylinder 34 in contact with the screen 64. The sample container (without the piston and weight therein) is then submerged in a 0.9 weight percent saline solution for a time period of about 60 minutes to saturate the absorbent structure. The same height measurements obtained for the Free Swell Gel Bed Permeability Test are taken, e.g., with the container 30 empty and with the absorbent structure sample within the container and saturated.

The absorbent structure permeability measurement is initiated by delivering a continuous flow of saline solution into the sample container 30 with the saturated absorbent structure, the piston 36, and the weight 48 inside. The saline solution is delivered to the container 30 at a flow rate sufficient to maintain a fluid height of about 7.8 cm (instead of the 4 cm used for the Free Swell Gel Bed Permeability Test) above the bottom of the sample container. The quantity of fluid passing through the absorbent structure versus time is measured gravimetrically. Data points are collected every second for at least twenty seconds once the fluid level has been stabilized to and maintained at about 7.8 cm in height. The flow rate Q through the absorbent structure sample 68 is determined in units of grams/second (g/s) by a linear least-square fit of fluid passing through the container (in grams) versus time (in seconds). The permeability of the absorbent structure is then determined using the equation set forth above for the Free Swell Gel Bed Permeability Test.

Where the Absorbent Structure Permeability Test is conducted as described above, and more particularly where the absorbent structure sample is submerged in the solution without the piston and weight thereon, the test is said to be conducted under “free swell” conditions whereby the absorbent structure is allowed to swell free of any restraining load, and may be referred to as the Free Swell ASP test. In a variation of this test, the piston and weight may be placed on the sample within the container and then the entire assembly can be submerged so that a load (e.g., approximately 0.3 psi) is applied to the sample as the sample becomes saturated and swells. When conducted in this manner the test is referred to as being conducted “under load,” and may be referred to as the 0.3 psi ASP test.

### Liquid Saturation Capacity Test

The following test is used to determine a saturation capacity of an absorbent structure. With reference to FIG. 4, an absorbent structure sample 308 having length and width dimensions of approximately four inches by four inches (approximately 10.16 cm by 10.16 cm) is weighed with any tissue wrap material on and the weight in grams is recorded. The sample 308 is then wrapped in paper toweling (not shown), such as Scott paper towel available from Kimberly-Clark Worldwide Inc. of Neenah, Wis., U.S.A., and submerged in an excess quantity of 0.9 weight percent sodium chloride solution in distilled water at room temperature (e.g., about 23 degrees Celsius) for about twenty minutes. After this time period, the sample 308 is removed from the test solution and placed on a test apparatus, indicated generally at 300 in FIG. 7, comprising a vacuum box 302, a TEFLON fiberglass screen 304 having 0.25 inch (0.6 cm) openings and supported by the vacuum box, and a flexible rubber cover 306 sized for overlaying the screen on the vacuum box.

More particularly, the absorbent structure sample 308 (with toweling) is placed uncovered (by the rubber cover) on the screen 304 and allowed to drip dry for about one minute. The rubber cover 306 is then placed over the sample 308 and screen 304 (e.g., to generally form a seal over the vacuum box 302) and a vacuum (V) of about 0.5 pounds/square inch (about 34.5 dynes/square cm) is drawn on the vacuum box (and hence the sample) for a period of about five minutes. The sample 308 is then removed from the apparatus and the toweling is taken off the sample, making an effort to recover loose fibers and superabsorbent particles along with the sample. The recovered sample is again weighed and the weight in grams is recorded. The saturation capacity of the sample is determined by subtracting the dry weight of the sample from the weight of the recovered sample after application of the vacuum and then dividing by the dry weight of the sample and is recorded as grams of liquid retained per gram of absorbent structure(g/g).

If absorbent structure fibers and/or superabsorbent material are drawn through the fiberglass screen into the vacuum box during testing, a screen having smaller openings should be used and the test should be re-done. Alternatively, a piece of tea bag material or other similar material can be placed between the sample and the screen and the total retention capacity adjusted for the liquid retained by the tea bag or other material.

At least three samples of each absorbent structure are tested and the results are averaged to provide the retention capacity (e.g., total and normalized retention capacity) of the absorbent structure.

### Void Volume Test

Void volume and compression recovery can be measured using an INSTRON or SINTECH tensile tester to measure the resisting force as a material is compressed between a movable platen and a fixed base at a constant rate using a certain amount of force and subsequently releasing the force at the same rate. Preferably pressure, or force, and platen position are recorded. If only force is recorded, pressure is calculated using:

$$P = \frac{F}{{A}_{p} \cdot 10 \text{,} 000 cm \text{/} {m}^{2}}$$

where:
P=pressure in Pascals
F=force pushing back on the platen in Newtons
Aₚ=area of the platen in square centimeters (18.9 cm²)

Void volume for a given platen position is calculated using the equation:

$$VV = \frac{( {x}_{o} - x ) \cdot {A}_{m} \cdot 0.1 cm \text{/} mm}{M} - \frac{1}{{ρ}_{component}}$$

where:
VV=void volume of material in cubic centimeters per gram
xₒ=initial platen position from the base in millimeters
x=platen position from initial position in millimeters
Aₘ=area of material in square centimeters
M=mass of-material in grams
ρ_{component}=constituent component density in grams per cubic centimeter

For webs made with multiple components (e.g., superabsorbent polymer and wood pulp fluff, or multiple types of superabsorbent polymer and/or wood pulp fluff), the web component density is the weight average of each individual component density:
ρ_{comp, Total}=wt % _{c ompl ρ compl}+wt %_{comp2} ρ_{comp2}+ . . .
where:
wt %=weight percent of the component type in the web or:

$$wt \% = \frac{component weight in composition}{total composition weight} \times 100 \%$$

The base must be larger in size than the platen. Zero height between platen and base distance is set by bringing the platen down until it barely touches the base. The platen is then raised to the desired initial height from the zero distance. The initial platen position must be greater than the initial thickness of the material so that the test starts out at zero pressure on the sample. The material can be the same size as the platen or larger.

One suitable compression tester is an INSTRON model 6021 with compression test software and 1 kN load cell made by Instron of Bucks, England. One suitable balance is a model PM4600, available from Mettler of Highstown, N.J.

For the purpose of measuring void volume, the following procedure should be used. A 4.9 cm diameter circular platen is used to compress materials against the base at a rate of 5.08 mm/min up to 909 gm load (4,690 Pascal or 0.68 pounds per square inch pressure). The platen is then returned at the same rate to the initial starting position. The initial starting position for the platen is 12.7 mm from the base. Material samples should be cut to 10.2 cm×10.2 cm and tested in the center. Force and position data should be recorded every 0.03 minutes or every 0.15 mm. Five repeats should be performed on separate material pieces.

### EXAMPLES

The following Examples describe various versions of the invention. Other versions within the scope of the claims herein will be apparent to one skilled in the art from consideration of the specification or practice of the invention as disclosed herein. It is intended that the specification, together with the Examples, be considered exemplary only, with the scope and spirit of the invention being indicated by the claims which follow the Examples.

### Example 1

This Example serves to demonstrate that superabsorbent materials having certain combinations of capacity and permeability are especially suitable for use in absorbent garments used by males, and superabsorbent materials having different combinations of capacity and permeability are especially suitable for use in absorbent garments used by females.

Prototype boy and girl training pants containing different superabsorbent materials were tested in a forced leakage study. Five different codes containing five different superabsorbent materials were tested for each gender. Each training pant included an absorbent pad comprising a substantially homogenous mixture of wood pulp fluff and superabsorbent material. The wood pulp used for all codes was ND416 available from Weyerhauser Corporation, Federal Way, Wash., U.S.A. The ratio (by weight) of superabsorbent material to pulp was 55.7% to 44.3%. Each absorbent pad exhibited a basis weight of approximately 700 grams per square meter (g/m²), and a density of approximately 0.41 grams per cubic centimeter (g/cm³). Each absorbent pad was a 405 millimeter by 100 millimeter rectangle. Each product included a surge material, 254 millimeters long and 62 millimeters wide, positioned 38 millimeters from the front waist edge of the absorbent pad. The surge material was a 50 grams per square meter, through-air-bonded, carded web comprised of a homogenous blend of 60 percent 3 denier type T-256 bicomponent fiber comprising a polyester core and polyethylene sheath and 40 percent 6 denier type T-295 polyester, both available from Kosa Corporation of Charlotte, N.C., U.S.A.

The five different superabsorbent materials included in this experiment and their respective key properties are listed in Table 1. Each superabsorbent material is available from Degussa Superabsorber of Greensboro, N.C., U.S.A. Superabsorbent properties tested included gel bed permeability (as described elsewhere herein) and centrifugal retention capacity (also described herein).

With the exception of the foregoing, each training pant otherwise resembled size 3T-4T Huggies® Pull-Ups® training pants, commercially available in 2002 from Kimberly-Clark Corporation, Neenah, Wis.

The study included 24 children, half boys and half girls. Subjects randomly tested one pant of each of the five codes in a forced leakage test. A small tube was secured to each subject, such that one opening of the tube was positioned near the male or female genitalia to correspond to the location at which the boy or girl would urinate into a training pant. The prototype training pant was then applied to the child, and the child wore a pair of cotton pants over the training pant. With the child in a prone position, 0.9 weight percent saline solution was introduced into the tube and thus into the pant in the quantity and order of 90 ml, 60 ml, 60 ml, 30 ml, 30 ml, and 30 ml until a leak larger than a 2.5 cm diameter circle appeared on the cotton pants, or until two hours had elasped. Fluid insults were introduced every 10 minutes until 300 grams of fluid had been added, after which the time between loadings was reduced to 5 minutes. Table 1 shows the properties of the superabsorbent materials and the results of the experiments. For both CRC and GBP tests, the superabsorbents were tested in virgin condition, described above. Methods for CRC and GBP are described elsewhere herein. “Mean L@L,” or “me an load at leak,” represents the mean fluid loading value, in grams, at which a group of products was observed to leak. “LD50” represents the fluid loading value below which 50% of the group of products was observed to leak. For both measures, values have been calculated separately for male and female wearer groups.

TABLE 1

| Super- | | | mean L@L | LD50 | mean L@L | LD50 |
|---|---|---|---|---|---|---|
| absorbent | CRC (g saline/g | GBP | male (g | male (g | female (g | female (g |
| material | superabsorbent) | (Darcys) | saline) | saline) | saline) | saline) |
| SP-1404 | 16.51 | 117.2 | 322.0 | 317.3 | 322.1 | 331.5 |
| SP-1405 | 19.66 | 92.4 | 336.6 | 333.0 | 317.5 | 317.6 |
| SP-1444 | 22.62 | 96.5 | 332.2 | 329.4 | 366.0 | 363.4 |
| SP-1407 | 29.23 | 32.9 | 314.6 | 312.0 | 351.9 | 353.4 |
| SXM9543 | 23 | 36.3 | 306.3 | 301.0 | 310.2 | 271.7 |

Mean load at leak and LD50 values were generally observed to increase with increasing superabsorbent capacity. When gender differences were considered, however, correlations between inherent superabsorbent permeability and product performance were observed to differ between male and female wearers.

FIGS. 5 and 6 show the relationships between female forced leakage testing results and two inherent properties of the superabsorbents being tested. FIG. 5 illustrates the relationship between mean load at leak and superabsorbent permeability, as measured using the GBP test. No trend between these properties is apparent for females. In contrast, FIG. 6 illustrates the relationship between mean load at leak and superabsorbent capacity, as measured by the CRC test. Surprisingly, as shown in the latter figure, a general correlation is observed in which increasing superabsorbent capacity is linked with higher forced leakage loadings.

FIGS. 7 and 8 illustrate comparable relationships for male forced leakage performance with SAP permeability and capacity, respectively. In the case of FIG. 7, a general positive correlation is observed between superabsorbent permeability and mean load at leak. The correlation between superabsorbent capacity and mean load at leak, as shown in FIG. 8, is weaker, if in fact a trend exists at all.

The relative numbers for LD50 in Table 1 fall into the same patterns as those observed for mean load at leak, providing additional corroboration of the observed trends. The foregoing results indicate that female product performance, at least as evinced by forced leakage testing, correlates more significantly with the inherent capacity of the constituent superabsorbent than with the superabsorbent's inherent permeability. In contrast, for males, product performance (again, as indicated by forced leakage testing) depends to a greater extent on superabsorbent permeability than is observed for females.

### Example 2

This Example provides further substantiation of the premise that superabsorbent materials having certain combinations of capacity and permeability are especially suitable for use in absorbent garments used by males, and superabsorbent materials having different combinations of capacity and permeability are especially suitable for use in absorbent garments used by females. This Example further demonstrates that the permeability of the overall absorbent core is more closely linked to the performance of absorbent garments used by males than to those used by females.

Prototype boy and girl training pants containing different superabsorbent materials were tested in an in-home use study. Five different codes containing five different superabsorbent materials were tested for each gender; Each training pant included an absorbent pad comprising a substantially homogenous mixture of wood pulp fluff and superabsorbent material. The wood pulp used for all codes was ND416 available from Weyerhauser Corporation, Federal Way, Wash., U.S.A. The amount of superabsorbent material and wood-pulp fluff in each product was approximately 14.2 grams and 10.5 grams, respectively. The superabsorbent/fluff mixture was distributed within each product such that the front half of each product contained approximately 60% by weight of the total superabsorbent/fluff mixture, and the back half of each product contained the remaining approximately 40% by weight of the total superabsorbent/fluff mixture. Each absorbent pad exhibited a density of approximately 0.36 grams per cubic centimeter (g/cm³). Each product included a surge material, 62 millimeters wide, with length and positioning selected for each gender as follows: male product surge was 254 millimeters long and was placed 38 millimeters behind the front waist edge of the absorbent pad, while female product surge was 203 millimeters long and was placed 77 millimeters behind the front waist edge of the absorbent pad. The surge material was a 50 grams permeability square meter, through-air-bonded, carded web comprised of a homogenous blend of 60 percent 3 denier type T-256 bicomponent fiber comprising a polyester core and polyethylene sheath and 40 percent 6 denier type T-295 polyester, both available from Kosa Corporation of Charlotte, N.C., U.S.A.

The five different superabsorbent materials included in this experiment and their respective key properties are listed in Table 2, along with selected performance information from the in-home testing. The last code listed, 2035-HP, is available from the Dow Chemical Corporation of Midland, Mich., USA; the other codes are experimental codes, obtained from Degussa Superabsorber of Greensboro, N.C., U.S.A. Codes SP1389 and SP1390 are common to a first technology“fami ly” of materials (Series A), and codes SP1393 and SP1394 are common to a second technology “fa mily” of materials (Series B) different from the first “fami ly,” wherein “famil y” has the meaning set forth earlier. As the physical data indicate, within each of the two Degussa technology families, the prototype superabsorbent materials were engineered to offer different combinations of capacity and permeability.

With the exception of the foregoing, each training pant otherwise resembled size 3T-4T Huggies® Pull-Ups® training pants commercially available in 2002.

The study included 260 children, roughly half boys and half girls. Subjects randomly tested each of the five codes for up to three days each. Leakage data for each training pant were collected from the participants. As in Example 1, “L D50” represents the urine loading value below which 50% of the group of products was observed to leak.

The CRC and GBP tests are explained above. In this Example, for both the CRC and GBP tests, the superabsorbents were tested after being “r eclaimed” from the absorbent composites, explained above. The GBP test was the Free Swell GBP test. The Absorbent Core Permeability was measured using a Free Swell ASP test.

TABLE 2

| | CRC | | | | | |
|---|---|---|---|---|---|---|
| Super- | (g | | Abs. Core | LD50 | LD50 | Technology |
| absorbent | saline/g | GBP | Permeability | Boy | Girl | Series or |
| Material | SAM) | (Darcys) | (Darcys) | (g urine) | (g urine) | “Fam ily” |
| SP1389 | 31.02 | 10.32 | 9.9 | 413.9 | 485.1 | A |
| SP1390 | 28.77 | 17.14 | 12.1 | 431.2 | 450.9 | A |
| SP1393 | 31.53 | 12.93 | 6.2 | 455.0 | 478.2 | B |
| SP1394 | 28.51 | 22.14 | 11.0 | 468.2 | 454.6 | B |
| 2035-HP | 26.78 | 19.12 | 15.5 | 419.2 | 466.6 | Control |

As evidenced by the data, graphically presented in FIGS. 9-12, SAM capacity appears to positively impact the performance of female products, whereas SAM permeability exhibits no positive correlation to female performance. In contrast, SAM capacity appears to have no positive correlation to male performance, while SAM permeability appears to correlate somewhat to male performance in a positive manner. Other factors may also influence male product performance more significantly than does superabsorbent capacity.

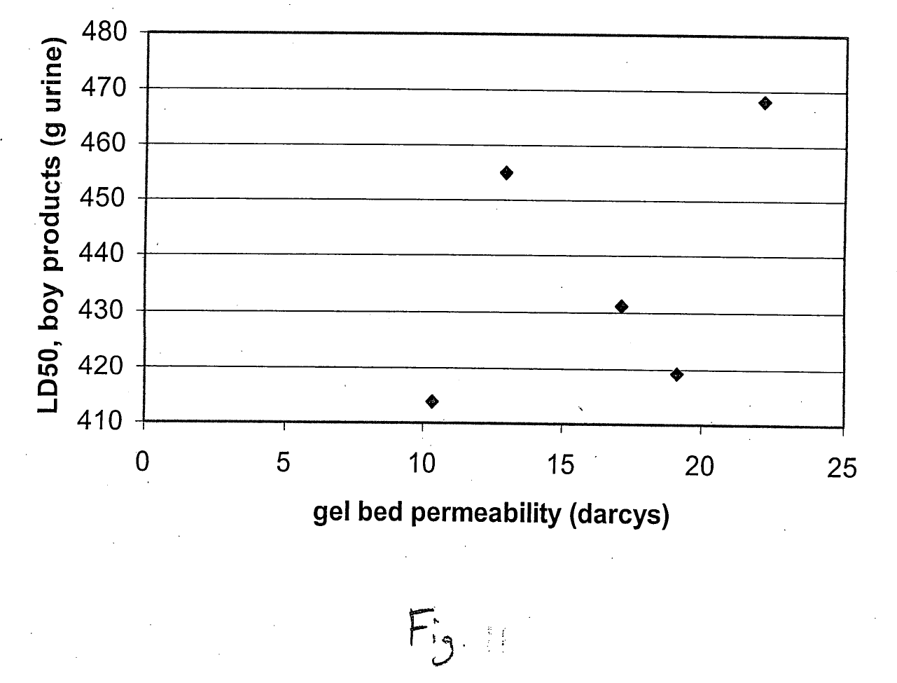

FIGS. 9 and 10 show the relationships between female performance and two inherent properties of the superabsorbents being tested. FIG. 9 illustrates the relationship between LD50 and superabsorbent permeability, as measured using the gel bed permeability test. These results suggest that increasing permeability does not enhance performance for girls; in fact, higher permeability appears in this example to degrade performance. In contrast, FIG. 10 illustrates the relationship between LD50 and superabsorbent capacity. In the latter figure, the data indicate that the higher capacity codes perform the best for girls. This observation holds true across both superabsorbent technology series, and is consistent with results from Example 1.

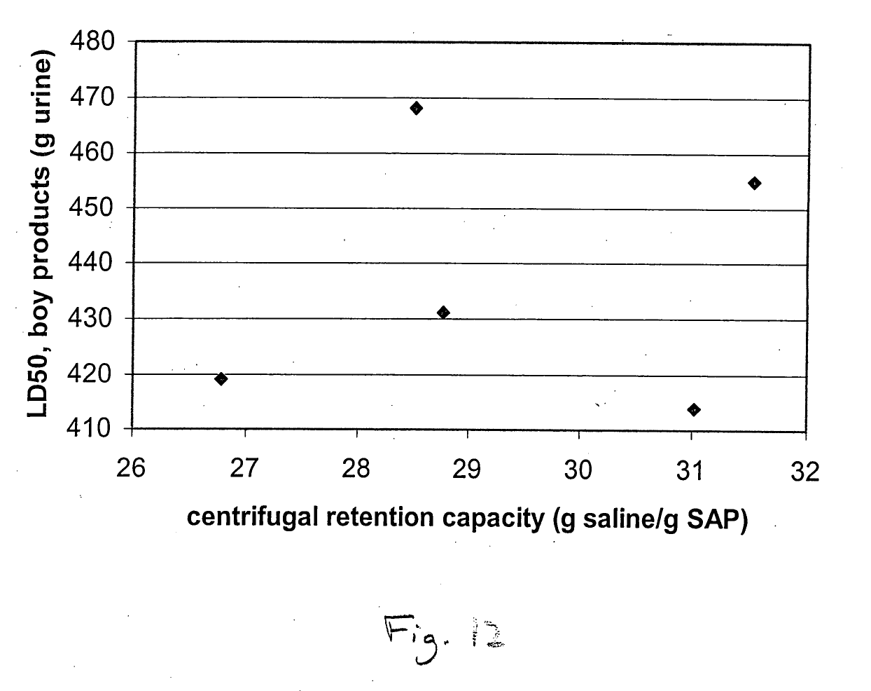

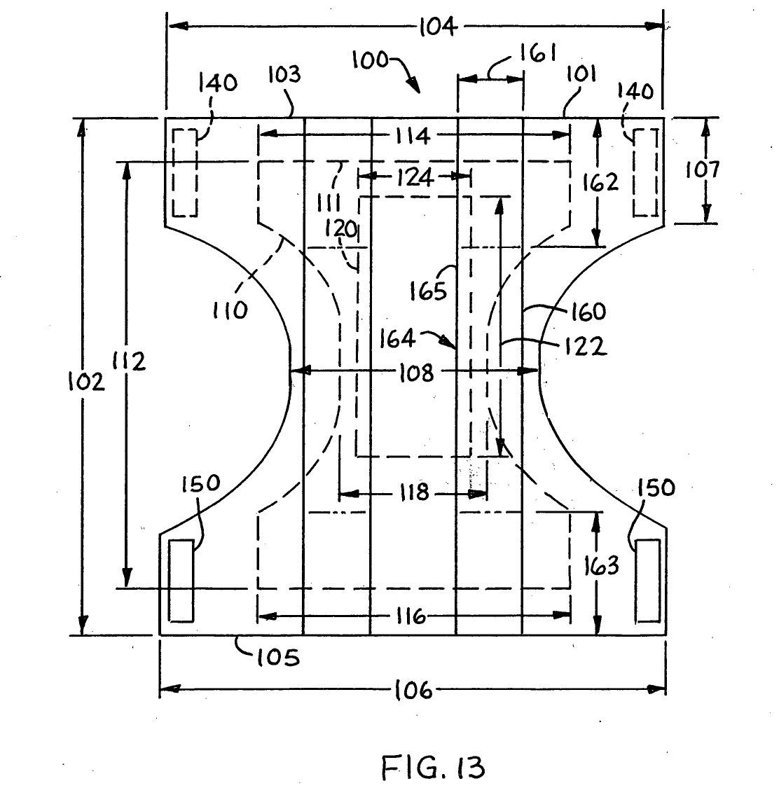

FIGS. 11 and 12 illustrate comparable relationships for male performance with SAP permeability and capacity, respectively. The results shown in FIG. 11 indicate that series B superabsorbents provide the best performance benefits for boys throughout the capacity range that was examined. Series B outperforms both the control code and series A codes, the latter of which possess equivalent capacities. This provides further substantiation that performance for boys depends on more than superabsorbent capacity alone. Moreover, FIG. 11 indicates that within each series, the higher permeability/lower capacity end of each series range provides the best performance. This observation can also be made on analysis of FIG. 12, which illustrates the capacity side of the relationship with LD50 values for males. This phenomenon is consistent with results from Example 1 that identified permeability as a more significant influence on performance for males than for females.

Finally, it appears that within a particular SAM technology family, an increase in the permeability of the overall absorbent core (i.e., fluff and SAM) had a positive effect on male performance.

### Example 3

Example 3 demonstrates that increasing the basis weight of a surge material has a relatively stronger positive impact on the performance of absorbent articles worn by boys than on those worn by girls. Example 3 further substantiates the premise that superabsorbent materials having certain combinations of capacity and permeability are especially suitable for use in absorbent garments used by males, and superabsorbent materials having different combinations of capacity and permeability are especially suitable for use in absorbent garments used by females.

Prototype boy and girl training pants containing different surge materials, superabsorbent polymers, and absorbent capacities were tested in a forced leakage study. Six different codes were tested by each gender. Each training pant (particular features of which are representatively illustrated in FIG. 13), included a stretchable main absorbent pad 110 comprising a substantially homogeneous mixture of 75% superabsorbent material, 10% cellulosic pulp, and 15% polymeric binder, respectively, where percentage amounts are by weight. With respect to the superabsorbent polymer in the main pad, all but one of the codes employed E1231-99, available from BASF Corporation, Portsmouth, Va., U.S.A. The remaining code (E) employed FAVOR SXM 9394, available from Stockhausen, Inc. Greensboro, N.C., U.S.A. With respect to the cellulosic pulp in the main pad, all of the codes employed Rayonier Sulfatate HJ, available from Rayonier, Jesup, Ga., U.S.A. With respect to the polymeric binder, all of the codes employed PLTD 1723, available from ExxonMobil Chemical Co, Houston, Tex., U.S.A. Each main absorbent pad 110 exhibited a basis weight of approximately 425 grams per square meter, and a thickness of approximately 1.5 millimeters. Each main absorbent pad 110 was generally hourglass in shape as representatively illustrated in FIG. 13, where the length 112 was 405 millimeters, the front width 114 and rear width 116 were both 143 millimeters, and the crotch width 118 was 60 millimeters.

Two of the codes (X and Z) included an additional, smaller, stretchable absorbent pad 120 positioned on the bodyside surface of the main absorbent pad. The purpose of the additional absorbent pad was to increase the overall absorbent capacity of the pant. Like the main absorbent pad, each additional smaller absorbent pad comprised a substantially homogeneous mixture of 75% superabsorbent material, 10% cellulosic pulp, and 15% polymeric binder, respectively, where percentage amounts are by weight. The superabsorbent polymer in each additional pad was E1231-99, available from BASF Corporation. The cellulosic pulp in each additional pad was Rayonier Sulfatate HJ. The polymeric binder in each additional pad was PLTD 1723. Each additional absorbent pad had a basis weight of approximately 425 grams per square meter, and a thickness of approximately 1.5 millimeters. Each additional absorbent pad was generally rectangular in shape as representatively illustrated in FIG. 13, where the length 122 was 280 millimeters, the width 124 was 57 millimeters, and the positioning from the front waist edge 111 of the main absorbent pad 110 was 25 millimeters. A process suitable for constructing such stretchable absorbent pads is described in U.S. Pat. No. 6,362,389 to McDowall et al. and assigned to Kimberly-Clark Corporation, the contents of which are hereby incorporated by reference to the extent consistent herewith.

The absorbent pad or pads of each code were sandwiched between a liquid-permeable bodyside liner and a liquid-impermeable outer cover. The liner was a 0.3 ounce per square yard (10 grams per square meter) polypropylene spunbond material, neck-stretched 35% to yield a resultant basis weight of 0.4 ounce per square yard, and treated with 0.35% by weight wetting agent; such a material is available from Kimberly-Clark Corporation, Dallas, Tex., U.S.A. The outer cover comprised an elastomeric 32 grams per square meter liquid-impermeable film laminated to a 20 grams per square meter extensible polypropylene spunbond. The liner and the outer cover shared the same dimensions and formed a pant chassis 101 having a length 102 of 479 millimeters, a front width 104 and a back width 106 of 300 millimeters, front and back panel lengths 107 of 80 millimeters, and a crotch width 108 of 100 millimeters. The pant 100 defined a front waist edge 103 and a back waist edge 105.

Each of the codes included a surge material (not shown). The surge material was a through-air bonded carded web, comprised of a homogeneous blend of 60% by weight of 1.5 denier bicomponent polyethylene sheath/polypropylene core fibers, and 40% by weight of 6 denier polyester fibers. The bicomponent fiber is available from FiberVision, Covington, Ga., U.S.A, and the polyester fibers are available from KoSa Corp., Charlotte, N.C., U.S.A. The completed surge material is available from Kimberly-Clark Corporation. The surge was 57 millimeters wide, 280 millimeters long, and positioned 25 millimeters from the front waist edge 111 of the main absorbent pad. Four of the codes (N, E, J, Z) employed a surge basis weight of 2.25 ounces per square yard (75 grams per square meter); the remaining two codes (X, V) employed a surge basis weight of 3.0 ounces per square yard (100 grams per square meter).

Each of the codes included a front and back waist elastic laminate (not shown). Each waist elastic laminate consisted of 7 strands of elastic thread spaced 4 millimeters apart, adhesively laminated between two pieces of spunbond. The elastic threads were laminated at 300 percent elongation, and were a 540 decitex Lyrca® material obtained from Invista Corp., Wilmington, Del., U.S.A. Each waist elastic laminate was transversely centered in the pant and abutted the corresponding waist edge, and was affixed to the pant using an ultrasonic bonder while stretch to an elongation of 66% from a relaxed state. In the final product, the waist elastic laminate was 275 millimeters long and 25.4 millimeters wide when measured at its 66% elongated state.

Each leg elastic laminate consisted of 4 strands of elastic thread spaced 3 millimeters apart, adhesively laminated between two pieces of spunbond, each of which were 12 millimeters wide. The elastic threads were laminated at 150 percent elongation and were a 540 decitex Lycra® material obtained from Invista Corp. One end of a piece of laminate 160 millimeters long (in the relaxed state) was positioned approximately 55 millimeters from the front waist edge of the outer cover and transversely inward approximately 20 millimeters from the side edge of the outer cover. The other end of the leg elastic laminate was positioned approximately 70 millimeters from the back waist edge of the outer cover and transversely inward approximately 20 millimeters from the side edge of the outer cover. The remainder of the leg elastic laminate was adhesively affixed to the pant such that it followed the leg contour of the pant with a uniform stretch, approximately 4 millimeters from the transverse edge of the outer cover. The laminate was thereby stretched approximately 150% from a relaxed state.

Codes J, V, X, and Z each included two containment flaps 160 constructed from the same stretchable laminate used for the outer cover, discussed above. Each flap 160 had a width 161 of approximately 54 millimeters and a length of 479 millimeters. The longitudinal ends of each flap was affixed to the liner across their entire width 161 throughout a front tacked down portion 162 fifty millimeters in length and a back tacked down portion 163 seventy-five millimeters in length. In the remaining, active portion 164 of the flap, the transversely inner most 27 millimeters of the flap remained unattached to the liner, resulting in a flap height of 27 millimeters in the crotch region of the pant. The remaining transversely outer most 27 millimeters of the flap was affixed to the liner. The flap employed a J-fold design, wherein the transversely inner most 27 millimeters of the flap comprised a J-fold. Three strands of elastic thread (540 decitex Lycra® material obtained from Invista Co.), spaced 3 millimeters apart, were adhesively laminated at 300% elongation within the distal edge 165 of the flap (i.e., within the J-fold) along the length of the flap, from a point 80 millimeters from the front waist edge 103 to a point 100 millimeters from the back waist edge 105. (Thus, the longitudinal ends of the flaps were not elasticized.) The distal edge 165 of each flap was spaced 18 millimeters from the longitudinal centerline of the pant.

Codes E and N each included two containment flaps identical to those described above as included in Codes J, V, X, and Z, with one exception. In codes E and N, the transversely innermost 12 inches of the flap was folded outward in a J-fold or C-fold manner (not shown) to transversely foreshorten the flap, akin that described in U.S. Pat. No. 5,895,382 to Popp et al. This outwardly folded portion was adhesively tacked down in the front of the pant along a strip 50 fifty millimeters in length and tacked down in the back of the pant along a strip 75 millimeters in length. In the remaining; active portion 164 of the flap, the outwardly folded portion was not adhesively tacked down, resulting in a flap height of 39 millimeters in the crotch region of the pant.

Each of the codes included a fastening system. A strip of hook material 140, 70 millimeters long and 16 millimeters wide, was positioned at each transversely outermost region of the front waist region on the garment side of the outer cover, as representatively illustrated in FIG. 13. The hook material was XKH-02-033, available from 3M Corporation, St. Paul, Minn., U.S.A. A strip of loop material 150, 75 millimeters long and 19 millimeters wide, was positioned at the transversely outermost regions of the back waist region on the bodyside of the liner. The loop material was Velcro® 3905, available from Velcro Group Corporation, Manchester, N.H., U.S.A.

Other than the absorbent pads, the various components of the pants were assembled together manually. Construction adhesive was used to affix the outer cover, the liner, the surge material, and the absorbent pads to each other. Two-sided adhesive tape was used to affixed the leg elastic laminate to the outer cover and the containment flaps to the liner. An ultrasonic bonder was used to affix the waist elastic laminate to the outer cover. Finally, the hook and loop fasteners were affixed to the outer cover using both construction adhesive and an ultrasonic bonder. Manners in which to obtain and use construction adhesive, two-sided tape, and ultrasonic bonders to manually assemble disposable absorbent article components are well known in the art and need not be recited in detail here.

The saturated capacity of each code was measured using the Liquid Saturation Capacity Test described above. Additionally, the permeability of the two superabsorbent materials was measured using the Free Swell Absorbent Structure Permeability test described above. The permeability of the FAVOR SXM 9394 superabsorbent material was 5 Darcies, while the permeability of the BASF E1231-99 superabsorbent material was approximately 250 Darcies.

The study included 12 boys and 12 girls in the weight range of 32-40 pounds. Subjects randomly tested one pant per code. A small tube was secured to each subject, such that one opening of the tube was positioned near the male or female genitalia to correspond to the location at which the boy or girl would urinate into a training pant. After being weighed in the dry state, the prototype training pant was then applied to the child, and the child wore a pair of cotton pants over the training pant. With the child in a prone position, 0.9 weight percent saline solution was introduced into the tube and thus into the pant in the quantity and order of 90 ml, 60 ml, 60 ml, 30 ml, 30 ml, and 30 ml until a leak larger than a 2.5 cm diameter circle appeared on the cotton pants. Fluid insults were introduced every 10 minutes until leakage was detected, or until 2 hours elapsed, whichever occurred earlier. Children were allowed to play in between fluid insults. When the pant was removed from the subject, the wet weight of the pant is recorded. Table 5 shows selected characteristics of the codes and the results of the experiment. “M ean L@L,” or “me an load at leak,” represents the mean fluid loading value, in grams, at which a group of products was observed to leak. Values have been calculated separately for male and female wearer groups.

TABLE 5

| | | Surge | | | | Mean |
|---|---|---|---|---|---|---|
| | SAM | basis | Additional | Pant | Mean | L@L |
| | permeability | weight | absorbent | capacity | L@L Boy | Girl |
| Code | (Darcies) | (osy) | pad | (g) | (g) | (g) |
| E | 5 | 2.25 | No | 423 | 164 | 216 |
| N | 250 | 2.25 | No | 401 | 244 | 211 |
| J | 250 | 2.25 | No | 401 | 210 | 312 |
| V | 250 | 3.00 | No | 401 | 290 | 308 |
| Z | 250 | 2.25 | Yes | 471 | 285 | 332 |
| X | 250 | 3.00 | Yes | 471 | 294 | 335 |

The data illustrate at least two trends. First, a comparison of code E to code N indicates that increasing SAM permeability enhances performance for boys, but has no corresponding effect on performance for girls. This is consistent with the trends observed in Examples 1 and 2. Second, a comparison of code J to code V indicates that increasing the basis weight of the surge material enhances performance for boys, but has no corresponding effect on performance for girls. This is consistent with the theory that permeability is more relevant to male performance than to female performance, as a higher basis weight surge provides more void volume to receive and distribute an initial insult of fluid. Stated another way, increasing the surge basis weight increases the permeability of the absorbent composite as a whole, and this increase yields an improved performance for boys, but not for girls. (A comparison of code Z to code X reveals a similar trend, though the improvement for boys may have been somewhat eclipsed by the enhanced absorbent capacity provided by the additional absorbent pad.)

Having described particular embodiments of the invention in detail, it will be readily apparent that various changes and modifications can be made without departing from the spirit of the invention. All of such changes and modifications are contemplated as being within the scope of the invention as defined by the appended claims and any equivalents thereto.

1. A method of marketing disposable absorbent articles for males and females, comprising:
providing a plurality of packages of male absorbent articles adapted for use by males, each male absorbent article comprising a first superabsorbent polymer;
providing a plurality of packages of female absorbent articles adapted for use by females, each female absorbent article comprising a second superabsorbent polymer,
wherein the first superabsorbent polymer and the second superabsorbent polymer exhibit different average permeabilites as measured by the Free Swell Gel Bed Permeability method; and
positioning the plurality of packages of male absorbent articles near the plurality of packages of female absorbent articles in a retail outlet.

2. The method of claim 2, wherein the average permeability of the first superabsorbent polymer is higher than the average permeability of the second superabsorbent polymer.

3. The method of claim 2, wherein the average permeability of the first superabsorbent polymer as measured in its reclaimed condition is at least about 40 Darcies.

4. The method of claim 3, wherein the average permeability of the second superabsorbent polymer as measured in its reclaimed condition is at most about 40 Darcies.

5. The method of claim 1, wherein the packages of male absorbent articles include indicia which denotes that the male absorbent articles include an absorbent material adapted for use in male absorbent articles.

6. The method of claim 1, wherein the packages of female absorbent articles include indicia which denotes that the female absorbent articles include an absorbent material adapted for use in female absorbent articles.

7. The method of claim 1, wherein each male absorbent article includes a surge material, and wherein no female absorbent articles include a surge material.

8. The method of claim 1, wherein the plurality of packages of male absorbent articles are positioned adjacent to the plurality of packages of female absorbent articles.

9. A method of marketing disposable absorbent articles for males and females, comprising:
providing a plurality of packages of male absorbent articles adapted for use by males, each male absorbent article comprising a first superabsorbent polymer;
providing a plurality of packages of female absorbent articles adapted for use by females, each female absorbent article comprising a second superabsorbent polymer,
wherein the first superabsorbent polymer and the second superabsorbent polymer exhibit different average capacities as measured by the Centrifugal Retention Capacity method; and
positioning the plurality of packages of male absorbent articles near the plurality of packages of female absorbent articles in a retail outlet.

10. The method of claim 9, wherein the average capacity of the first superabsorbent polymer is lower than the average capacity of the second superabsorbent polymer.

11. The method of claim 10, wherein the average capacity of the first superabsorbent polymer is at most about 28 g/g.

12. The method of claim 11, wherein the average capacity of the second superabsorbent polymer is at least about 27 g/g.

13. The method of claim 9, wherein the packages of male absorbent articles include indicia which denotes that the male absorbent articles include an absorbent material adapted for use in male absorbent articles.

14. The method of claim 9, wherein the packages of female absorbent articles include indicia which denotes that the female absorbent articles include an absorbent material adapted for use in female absorbent articles.

15. The method of claim 9, wherein each male absorbent article includes a surge material, and wherein no female absorbent articles include a surge material.

16. The method of claim 9, wherein the plurality of packages of male absorbent articles are positioned adjacent to the plurality of packages of female absorbent articles.

17. A method of marketing disposable absorbent articles for males and females, comprising:
providing a plurality of packages of male absorbent articles adapted for use by males, each male absorbent article comprising a first superabsorbent polymer, the first, superabsorbent polymer exhibiting a first average permeability and a first average capacity;
providing a plurality of packages of female absorbent articles adapted for use by females, each female absorbent article comprising a second superabsorbent polymer, the second superabsorbent polymer exhibiting a second average permeability and a second average capacity,
wherein the average permeabilities are measured by the Free Swell Gel Bed Permeability method, and the average capacities are measured by the Centrifugal Retention Capacity method,
wherein a first ratio of the first average permeability to the first average capacity is higher than a second ratio of the second average permeability to the second average capacity; and
positioning the plurality of packages of male absorbent articles near the plurality of packages of female absorbent articles in a retail outlet.

18. The method of claim 17, wherein the first ratio is at least about 1.6 Darcy-g/g.

19. The method of claim 17, wherein the second ratio is at most about 1.6 Darcy-g/g.

20. The method of claim 17, wherein the first ratio is at least about 30% greater than the second ratio.

21. The method of claim 17, wherein the first ratio is at least about 70% greater than the second ratio.

22. A method of marketing disposable absorbent articles for males and females, comprising:
providing a plurality of packages of male absorbent articles adapted for use by males, each male absorbent article comprising a first superabsorbent polymer;
providing a plurality of packages of female absorbent articles adapted for use by females, each female absorbent article comprising a second superabsorbent polymer,
wherein the first superabsorbent polymer exhibits a first average permeability as measured by the Free Swell Gel Bed Permeability method, and the second superabsorbent polymer exhibits an average permeability as measured by the Free Swell Gel Bed Permeability method, wherein a ratio of the first average permeability to the second average permeability is at least about 1.2; and
positioning the plurality of packages of male absorbent articles near the plurality of packages of female absorbent articles in a retail outlet.

23. The method of claim 22, wherein the ratio of the first average permeability to the second average permeability is at least about 1.6.

24. The method of claim 22, wherein the packages of male absorbent articles include indicia which denotes that the male absorbent articles include an absorbent material adapted for use in male absorbent articles.

25. The method of claim 22, wherein the packages of female absorbent articles include indicia which denotes that the female absorbent articles include an absorbent material adapted for use in female absorbent articles.

26. The method of claim 22, wherein each male absorbent article includes a surge material, and wherein no female absorbent articles include a surge material.

27. The method of claim 22, wherein the plurality of packages of male absorbent articles are positioned adjacent to the plurality of packages of female absorbent articles.

28. A method of manufacturing disposable absorbent articles for males and females, comprising:
assembling a plurality of male absorbent articles adapted for use by males, each male absorbent article comprising a first superabsorbent polymer;
assembling a plurality of female absorbent articles adapted for use by females, each female absorbent article comprising a second superabsorbent polymer,
wherein the first superabsorbent polymer and the second superabsorbent polymer exhibit different average permeabilities as measured by the Free Swell Gel Bed Permeability method;
placing the plurality of male absorbent articles into a first package, wherein the first package includes indicia denoting that the absorbent articles therein are adapted for use by males; and
placing the plurality of female absorbent articles into a second package, wherein the second package includes indicia denoting that the absorbent articles therein are adapted for use by females.

29. The method of claim 28, wherein the average permeability of the first superabsorbent polymer is higher than the average permeability of the second superabsorbent polymer.

30. The method of claim 29, wherein the average permeability of the first superabsorbent polymer is at least about 40 Darcies.

31. The method of claim 30, wherein the average permeability of the second superabsorbent polymer is at most about 40 Darcies.

32. The method of claim 28, wherein each male absorbent article includes a surge material, and wherein no female absorbent articles include a surge material.

33. A method of manufacturing disposable absorbent articles for males and females, comprising:
assembling a plurality of male absorbent articles adapted for use by males, each male absorbent article comprising a first superabsorbent polymer;
assembling a plurality of female absorbent articles adapted for use by females, each female absorbent article comprising a second superabsorbent polymer,
wherein the first superabsorbent polymer and the second superabsorbent polymer exhibit different average capacities as measured by the Centrifugal Retention Capacity method;
placing the plurality of male absorbent articles into a first package, wherein the first package includes indicia denoting that the absorbent articles therein are adapted for use by males; and
placing the plurality of female absorbent articles into a second package, wherein the second package includes indicia denoting that the absorbent articles therein are adapted for use by females.

34. The method of claim 33, wherein the average capacity of the first superabsorbent polymer is lower than the average capacity of the second superabsorbent polymer.

35. The method of claim 34, wherein the average capacity of the first superabsorbent polymer is at most about 28 g/g.

36. The method of claim 35, wherein the average capacity of the second superabsorbent polymer is at least about 27 g/g.

37. The method of claim 33, wherein each male absorbent article includes a surge material, and wherein no female absorbent articles include a surge material.

38. A method of manufacturing disposable absorbent articles for males and females, comprising:
assembling a plurality of male absorbent articles adapted for use by males, each male absorbent article comprising a first superabsorbent polymer;
assembling a plurality of female absorbent articles adapted for use by females, each female absorbent article comprising a second superabsorbent polymer,
wherein the first superabsorbent polymer exhibits a first average permeability as measured by the Free Swell Gel Bed Permeability method, and the second superabsorbent polymer exhibits an average permeability as measured by the Free Swell Gel Bed Permeability method, wherein a ratio of the first average permeability to the second average permeability is at least about 1.2;
placing the plurality of male absorbent articles into a first package, wherein the first package includes indicia denoting that the absorbent articles therein are adapted for use by males; and
placing the plurality of female absorbent articles into a second package, wherein the second package includes indicia denoting that the absorbent articles therein are adapted for use by females.

39. The method of claim 38, wherein the ratio of the first average permeability to the second average permeability is at least about 1.6.

40. The method of claim 38, wherein the packages of male absorbent articles include indicia which denotes that the male absorbent articles include an absorbent material adapted for use in male absorbent articles.

41. The method of claim 38, wherein each male absorbent article includes a surge material, and wherein no female absorbent articles include a surge material.
